(19) United States
(12) Patent Application Publication
(10) Pub. No.: US 2026/0351405 A1
(43) Pub. Date: Nov. 5, 2026

(54) DISEASE CHARACTERISATION

(71) Applicant: Roche Molecular Systems, Inc., Pleasanton, CA (US)

(72) Inventors: Eldad KLAIMAN, Starnberg (DE); Diane DUROUX, Liège (BE); Ofir ETZ HADAR, Penzberg (DE); Jacob GILDENBLAT, Holon (IL); Michael KING, München (DE); Kristel VAN STEEN, Temse (Oost-Vlaanderen) (BE); Antoaneta Petkova VLADIMIROVA, Mountain View, CA (US); Christian WOHLFART, Penzberg (DE)

(21) Appl. No.: 19/120,678

(22) PCT Filed: Oct. 12, 2023
(86) PCT No.: PCT/EP2023/078390

§ 371 Date: Apr. 11, 2025

Related U.S. Application Data
(60) Provisional application No. 63/379,155, filed on Oct. 12, 2022.

(51) Int. Cl. G16H 50/20; G16H 50/50

(52) U.S. Cl. CPC G16H 50/20; G16H 50/50

(57) ABSTRACT

Computer-implemented methods of providing a disease diagnosis or prognosis for a patient are described. These comprise generating individual networks, each individual network comprising a plurality of nodes and edges between pairs of the nodes, wherein each node is indicative of a biological factor in biological data for an individual, and each edge is indicative of a relationship between a pair of biological factors corresponding to the nodes that the edge connects for the respective individual; determining the value of one or more similarity metrics between one or more individual networks generated for the patient and one or more individual networks generated for other individuals in the plurality of individuals; and predicting a diagnosis or prognosis for the patient using a machine learning model that takes as input the values of one or more similarity metrics.

### FIELD OF THE DISCLOSURE

The present invention relates to methods for analysing biological samples from subjects, using machine learning and subject-specific graphs representing a plurality of biological factors and relationships between biological factors. In particular, the present invention relates to methods for providing a prognosis, diagnosis, therapeutic recommendation, or patient selection, using such methods, and to related systems and devices.

### BACKGROUND

Disease subtyping refers to the identification of homogeneous groups of patients. It can be used to detect a disease's severity or target treatments with the highest probability of success. Disease subtyping is essential in cancer research since cancers are highly diverse in molecular types and severity. Many methods for disease subtyping analyses rely on a single data modality only. However, one modality is unlikely to be informative enough to capture the whole complexity of complex diseases. In addition, a large panel of data is available, making multi-modality integration realistic. For instance, multiple studies investigated the benefits of combining images, and genomic data (Ash et al. 2021; Schneider et al. 2022).

Data from multiple modalities can be integrated at different stages of a predictive method. Using late integration, the data sources are independently used to obtain a classification, and the classification results are then merged. The main disadvantages of this method are that it does not take advantage of the possible complementarity of the modalities. Alternatively, using early fusion, the data from multiple data sources can be concatenated before applying a machine-learning model. Whereas this solution is simple to implement, concatenation may decrease the signal-to-noise ratio in each data modality. In the last decade, alternative have been proposed to combine data between the start and end steps to solve these issues.

For example, iCluster (Shen et al. 2009) applies data fusion and dimensionality reduction at the same time to multiple genomic data types. This method uses a Gaussian latent variable model (i.e. jointly estimating latent tumour subtypes from different genomic modalities assuming to each be linearly related to the latent variable through a respective model) with lasso-type penalty terms to induce sparsity in the coefficient matrices toward feature selection. One drawback of this approach is its high computational complexity. An alternative is Affinity Aggregation for Spectral Clustering (Huang et al. 2012). The main idea is to compute a matrix of similarity between samples, for each data source. Then, these multiple affinity matrices are clustered via Spectral Clustering using linear combination with weights optimised using multiple kernel learning. Similarly, Similarity Network Fusion (SNF) (Wang et al. 2014) was implemented to combine multiple similarity matrices into a single one by iteratively updating the matrices to make them more and more similar until the algorithm converges. This final matrix becomes the new input to the classification algorithm. Later, regularised unsupervised multiple kernel learning was introduced (Speicher and Pfeifer, 2015). This extends multiple kernel learning for dimensionality reduction (projecting samples into a lower dimensional subspace for further analysis) by adding a constraint that leads to regularisation of the vector controlling the kernel combinations to avoid overfitting during optimisation.

Despite these advances, there is still a need for improved methods for analysing biological data to characterise a disease in a subject.

### SUMMARY OF THE DISCLOSURE

Broadly, the present inventors postulated that none of the existing methods for personalised disease characterisation make use of the full information contained in biological datasets, at least because they consider only one variable at a time and do not account for interactions between variables. The inventors postulated that this could be addressed by using networks (graphs) to consider interactions between pairs of features. They further postulated that in this context it would be beneficial to use individual (i.e. subject-specific) networks rather than cohort based networks, because such networks represent individual relations between variables for each person and are therefore particularly useful for precision medicine such as disease subtyping. The inventors therefore developed a multi-step pipeline to predict outcomes via graphs. First, one or more networks are constructed for each individual from raw biological data about the subject. Then the distance between each pair of subject-specific graphs is calculated to estimate how similar individuals are. The results are gathered in a similarity matrix (individual-to-individual similarity) that becomes the input of the machine learning model. In other words, the inventors consider as a new variable the similarities to a reference panel. Then, the outcome is predicted from these similarities toa reference set. The inventors demonstrated that graph-based methods for prediction of disease subtype and severity achieved competitive or improved performance with methods that consider raw data not in a graph-based context. They further showed that the process is advantageously able to flexibly integrate multiple modalities with different characteristics, and that intermediate integration is often advantageous for this.

Accordingly, a first aspect provides a method of characterising a disease in a patient, the method comprising: obtaining, for each of a plurality of individuals comprising the patient, biological data comprising values for a plurality of biological factors; generating, for each of the plurality of individuals, one or more individual networks, each individual network comprising a plurality of nodes and edges between pairs of the nodes, wherein each node is indicative of a biological factor in the biological data for an individual, and each edge is indicative of a relationship between a pair of biological factors corresponding to the nodes that the edge connects for the respective individual; determining the value of one or more similarity metrics between one or more individual networks generated for the patient and one or more individual networks generated for other individuals in the plurality of individuals; and predicting a diagnosis or prognosis for the patient using a machine learning model configured to predict a diagnosis or prognosis of the disease in the patient, wherein the machine learning model has been trained to take as input the values of one or more similarity metrics between individual networks and produces as output a diagnosis or prognosis.

The methods according to the present aspect may have one or more of the following optional features.

Determining the value of one or more similarity metrics may comprise determining the value of one or more similarity matrices each comprising the values of a similarity metric between individual networks of pairs of the plurality of individuals.

The one or more similarity metrics may comprise, for each of a plurality of pairs of individual networks, a similarity between edges in the individual networks, a similarity between edges in the individual networks and a similarity between nodes in the individual network, or a similarity combining a similarity between edges in the individual networks and a similarity between nodes in the individual network.

Each node in an individual network may have a value that is the value of a biological factor in the biological data for the respective individual. Each edge in an individual network may have a value that is the product of the values of the nodes that it connects for the respective individual. Each edge in an individual network may have a value that is the difference between the edge value for a network obtained using the plurality of individuals without or without the respective individual. Each edge in an individual network may have a value e^{x}ᵢⱼ=N*(e^{α}ᵢⱼ−e^{α-x}ᵢⱼ)+e^{α-x}ᵢⱼ, where e^{α}ᵢⱼ is the weight of an edge between nodes i and j in a network modeled on all N individual of the plurality of individuals and e^{α-x}ᵢⱼ is the weight of that edge in a network modeled on all samples except the respective individual x.

The one or more similarity metrics may comprise, for each of a plurality of pairs of individual networks, a similarity between nodes in the individual networks obtained as a Spearman correlation coefficient, an affinity matrix, or a Gaussian kernel using a distance metric between vectors corresponding to the nodes in the respective individuals. The one or more similarity metrics may comprise, for each of a plurality of pairs of individual networks, a similarity combining a similarity between edges in the individual networks and a similarity between nodes in the individual network obtained as a Spearman correlation coefficient, an affinity matrix, or a Gaussian kernel using a distance metric between vectors corresponding to the nodes in the respective individuals. The one or more similarity metrics may comprise, for each of a plurality of pairs of individual networks, a similarity between nodes in the individual networks obtained as a Spearman correlation coefficient, or a similarity combining a similarity between edges in the individual networks and a similarity between nodes in the individual network obtained as a Spearman correlation coefficient. The one or more similarity metrics comprise, for each of a plurality of pairs of individual networks, a similarity between edges in the individual networks obtained as an Euclidian distance, Jaccard distance, edge difference distance, DeltaCon distance, spectral distances, graphlet-based measures, Hamming distance, Shortest path kernel, k-step random walk kernel, graph diffusion distance and Portrait Divergence.

The one or more similarity metrics comprise, for each of a plurality of pairs of individual networks, a similarity combining a similarity between nodes in the individual networks and a similarity between edges in the individual network obtained as an Euclidian distance, Jaccard distance, edge difference distance, DeltaCon distance, spectral distances, graphlet-based measures, Hamming distance, Shortest path kernel, k-step random walk kernel, graph diffusion distance and Portrait Divergence.

The one or more similarity metrics may comprise, for each of a plurality of pairs of individual networks, a similarity between edges in the individual networks obtained as an edge difference distance, or a similarity combining a similarity between nodes in the individual networks and a similarity between edges in the individual network obtained as an edge difference distance. An edge difference distance may be obtained as the Frobenius norm of the difference between a pair of matrices comprising the values of the edges in the respective individual networks for which a similarity is obtained.

The method may further comprise generating a report of the diagnosis or prognosis of the disease in the patient. The method may further comprise generating the machine learning model configured to predict a diagnosis or prognosis of the disease in patients.

The biological data for each of a plurality of individuals may comprise values for a plurality of biological factors comprising a plurality of sets of factors obtained using respective data modalities, wherein the biological data comprises biological data obtained using a plurality of data modalities. The biological data for each of the plurality of individuals may comprise values for a plurality of biological factors derived from at least one of transcriptomics, proteomics, metabolomics, microbiome, clinical, medical imaging, demographic or histopathology data, optionally wherein the biological data for each of the plurality of individuals comprises values for a plurality of biological factors derived from transcriptomic or proteomic data and values for a plurality of biological factors obtained from histopathology data.

Obtaining for each of the plurality of individuals, one or more individual networks, may comprise obtaining for each of the plurality of individuals at least one individual network using values for a plurality of biological factors that comprise biological factors obtained using at least two different data modalities.

The one or more similarity metrics between one or more individual networks may comprise one or more similarity metrics derived from individual networks that are obtained from data comprising values of biological factors obtained using at least two different data modalities. Obtaining for each of the plurality of individuals, one or more individual networks, may comprise obtaining for each of the plurality of individuals, a plurality of individual networks, each individual network being obtained using values for a respective plurality of biological factors, optionally wherein each individual network is obtained using values for a respective plurality of biological factors obtained using the same data modality, and the plurality of individual networks comprise individual networks obtained using at least two different data modalities.

The one or more similarity metrics between one or more individual networks may comprise a first set of one or more similarity metrics derived from individual networks that are obtained from data comprising values of biological factors obtained using a first set of data modalities, and a second set of one or more similarity metrics derived from individual networks that are obtained from data comprising values of biological factors obtained using a second set of data modalities, wherein the first set is different from the second set. The one or more similarity metrics between one or more individual networks may comprise one or more similarity metrics obtained by combining, for a pair of individuals, a plurality of similarity metrics each derived from a pair of individual networks for the respective individuals obtained from data comprising values of biological factors obtained using a different set of one or more data modalities.

The machine learning model may comprise a plurality of machine learning models, each machine learning model configured to predict a diagnosis or prognosis of the disease in the patient, wherein each machine learning model has been trained to take as input the values of a respective subset of the one or more similarity metrics between individual networks and produce as output a diagnosis or prognosis, wherein the respective subsets of similarity metrics are derived from individual networks that are generated from values of biological factors obtained using respective data modalities, and wherein providing a diagnosis or prognosis for the patient comprises combining the outputs of the plurality of machine learning models.

The machine learning model comprises a classification or a regression model. The machine learning model may comprise a support vector machine model.

Providing a diagnosis or prognosis for the patient may comprise combining predicting a disease subtype or severity. The disease may be cancer. Providing a diagnosis or prognosis for the patient may comprise predicting a Gleason score for a patient diagnosed as having prostate cancer, classifying a patient diagnosed as having brain cancer between a first class corresponding to brain lower grade glioma (Igg) and a second class corresponding to gliobastoma multiforme (gbm), or classifying a patient diagnosed as having lung cancer between a first class corresponding to lung adenocarcinoma (luad) and a second class corresponding to lung squamous cell carcinoma (lusc).

The biological factors may comprise gene or protein expression levels and optionally histopathology data. In embodiments, the disease is prostate cancer and the biological factors comprises an expression level for MAP7. In embodiments, the disease is brain cancer and the biological factors comprises an expression level for GTP2 and/or HIPK2. In embodiments, the disease is lung cancer and the biological factors comprises an expression level for TGM2 and/or DUSP4.

The biological factors may comprise latent variables of a trained machine learning model applied to image data, optionally wherein the image data is histopathology data. The trained machine learning model may be a machine learning model, optionally a neural network, that has been trained in a supervised manner to take as input histopathology data and provide as output a disease type label.

At least one of the one or more individual networks, optionally all of the one or more individual networks, may comprise nodes that have been selected using a feature selection process and/or edges that have been selected using a feature selection process. Generating, for each of the plurality of individuals, one or more individual networks may comprise applying a feature selection process to a plurality of nodes each indicative of a biological factor in the biological data for an individual, and/or applying a feature selection process to a plurality of edges is indicative of a relationship between a pair of biological factors corresponding to the nodes that the edge connects for the respective individual.

Generating, for each of the plurality of individuals, one or more individual networks may comprise selecting a plurality of nodes each indicative of a biological factor in the biological data for an individual for inclusion in each respective individual network, wherein the selection is performed separately for each individual or collectively for the plurality of individuals. Selecting a plurality of nodes may comprise selecting a plurality of biological factors that are different between an individual and a reference set of individuals or selecting a plurality of nodes that have a variability across the plurality of individuals that satisfies one or more predetermined criteria.

Generating, for each of the plurality of individuals, one or more individual networks may comprise selecting a plurality of edges indicative of a relationship between a pair of biological factors corresponding to the nodes that the edge connects for the respective individual, wherein the selection is performed separately for each individual or collectively for the plurality of individuals. Selecting a plurality of edges may comprise selecting a plurality of edges that are different between an individual and a reference set of individuals or selecting a plurality of edges that are different between a plurality of subsets of the plurality of individuals.

Generating, for each of the plurality of individuals, one or more individual networks may comprise selecting a plurality of nodes each indicative of a biological factor in the biological data for an individual for inclusion in each respective individual network, wherein selecting a plurality of nodes comprises selecting a plurality of nodes that have a variability across the plurality of individuals that satisfies one or more predetermined criteria.

Generating, for each of the plurality of individuals, one or more individual networks may comprise selecting a plurality of edges indicative of a relationship between a pair of biological factors corresponding to the nodes that the edge connects for the respective individual, wherein selecting a plurality of edges comprises selecting a plurality of edges that are associated with a difference between (a) a first edge value obtained for a pair of nodes for a first subset of the plurality of individuals, and (b) a second edge value obtained for the same pair of nodes for a second subset of the plurality of individuals, the difference satisfying a predetermined criterion. The first edge value may be the correlation between the pair of nodes across the first subset of the plurality of individuals and the second edge value may be the correlation between the pair of nodes across the second subset of the plurality of individuals. The predetermined criterion may be the difference being amongst a predetermined threshold or amongst the top x differences amongst all possible edges between nodes in the individual networks, optionally after node selection, wherein x is a predetermined value, e.g. 3, 5, 10.

The methods described herein are computer implemented unless context indicates otherwise. Indeed, the size of biological data (e.g. omics data) typically used for the purpose of this method, and the size of networks to be compared (typically comprising multiple hundreds of nodes and hundreds to thousands of edges), means that the process of determining the similarity between networks and training a machine learning model to classify subjects based on these similarities is of a complexity that places the methods described herein far beyond the capability of mental investigation.

Thus, also described according to the first aspect is a method of diagnosing a disease of a patient, the method comprising:
obtaining biological factors of a plurality of individuals, at least some of the biological factors related to a disease;
for each of the plurality of individuals, generating an individual graph of nodes and edges between the nodes, each node correlating to one of the biological factors and wherein edges between the nodes correlate to relationships between the biological factors for the respective individual;
calculating one or more similarity matrices representing the similarity between the individual graphs;
generating a machine learning model configured to predict a diagnosis or prognosis of the disease in patients, the machine learning model trained with the one or more similarity matrices and biological factors;
based on the machine learning model and on biological factors obtained from a patient, predicting a diagnosis or prognosis of the disease in the patient; and
generating a report of the diagnosis or prognosis of the disease in the patient.

The one or more similarity matrices may be generated from similarities between the individual graphs and similarities between biological factors independent of the graphs. The one or more similarity matrices may be based on Spearman calculations and at least one of a node product or lioness calculations. The biological factors on which the similarity matrices are based may comprise at least one gene or protein expression and histopathology reading, and predicting the diagnosis or prognosis of the disease may comprise predicting a cancer diagnosis or prognosis. Predicting the cancer diagnosis or prognosis may comprise determining at least one of a type or severity of cancer. Determining the at least one of the type or severity of cancer may comprise calculating a Gleason score. Determining the at least one of the type or severity of cancer may comprise distinguishing between brain lower grade glioma (Igg) and gliobastoma multiforme (gbm). Determining the at least one of the type or severity of cancer may comprise distinguishing between lung adenocarcinoma (luad) and lung squamous call carcinoma (lusc). The cancer diagnosis or prognosis may comprise a prostate cancer and the at least one gene or protein expression may comprise MAP7. The cancer diagnosis or prognosis may comprise a brain cancer and the at least one gene or protein expression may comprise GTP2 or HIPK2. The cancer diagnosis or prognosis may comprise a lung cancer and the at least one gene or protein expression may comprise TGM2 or DUSP4.

According to a second aspect, there is provided a computer-implemented method for obtaining a tool for characterising a disease in a patient, the method comprising: obtaining, for each of a plurality of training individuals biological data comprising values for a plurality of biological factors for the individual, and a diagnosis or prognosis label associated with the individual; generating, for each of the plurality of individuals, one or more individual networks, each individual network comprising a plurality of nodes and edges between pairs of the nodes, wherein each node is indicative of a biological factor in the biological data for an individual, and each edge is indicative of a relationship between a pair of biological factors corresponding to the nodes that the edge connects for the respective individual; determining the value of one or more similarity metrics between one or more individual networks generated for the patient and one or more individual networks generated for other individuals in the plurality of individuals; and generating a machine learning model configured to predict a diagnosis or prognosis of the disease in a patient, wherein the machine learning model takes as input the values of the one or more similarity metrics between individual networks and produces as output a diagnosis or prognosis. The method according to the present aspect may have any of the features described in relation to the first aspect. In particular, the method may comprise any of the steps described herein in relation to methods of characterising a disease in a patient, such as feature selection steps, steps of obtaining biological factors for example from imaging data, steps of obtaining similarity metrics, steps of obtaining individual networks, etc.

According to a third aspect, there is provided a computer-implemented method for providing a treatment recommendation for a patient with a disease, the method comprising: characterising the disease in the patient using the method of any embodiment of the first aspect, and selecting the patient for treatment with a treatment associated with the predicted diagnosis or prognosis. The method may further comprise treating the patient with the selected treatment.

According to any aspect, obtaining biological data comprising values for a plurality of biological factors may comprise receiving data from a database, computer-readable memory, or user interface. According to any aspect, obtaining biological data comprising values for a plurality of biological factors may comprise measuring the values of one or more biological factors in a sample previously obtained from an individual.

According to a fourth aspect, there is provided a computer-implemented method of performing quality control for biological data about a patient with a disease, the method comprising: characterising the disease in the patient using the method of any embodiment of the first aspect using biological data about the patient comprising values for a plurality of subsets of biological factors obtained using respective different data modalities; characterising the disease in the patient using the method of any embodiment of the first aspect using biological data about the patient comprising only values for a first subset of biological factors; and comparing the predicted diagnosis or prognosis obtained using the plurality of subsets of biological factors and the first subset of biological factors, wherein a predicted diagnosis or prognosis being different for the first subset of biological factors compared to the plurality of subsets of biological factors is indicative of poor quality of the biological data comprising the first subset of biological factors.

According to a further aspect, there is provided a system comprising: a processor; and a computer readable medium comprising instructions that, when executed by the processor, cause the processor to perform the (computer-implemented) steps of the method of any preceding aspect. According to a further aspect, there is provided a non-transitory computer readable medium or media comprising instructions that, when executed by at least one processor, cause the at least one processor to perform the method of any embodiment of any aspect described herein. According to a further aspect, there is provided a computer program comprising code which, when the code is executed on a computer, causes the computer to perform the method of any embodiment of any aspect described herein.

Embodiments of the present invention will now be described by way of example and not limitation with reference to the accompanying figures. However various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 is a flowchart illustrating a method of characterising a disease in a subject as described herein.

FIG. 2 shows an embodiment of a system for characterising a disease in a subject as described herein.

FIG. 3 illustrates schematically a data integration and prediction workflow used in examples of the disclosure.

FIG. 4 shows results of assessment of classification performance (macro F1 scores (%)) obtained for classifications using PPNs from INs, built using different multi-modality approaches. A. Classification of lung cancer severity. B. Classification of brain cancer types. C classification of lung cancer types. The greener, the better the prediction, and the redder, the worse the prediction. Untested approaches are left blank. The three first rows refer to approaches based on the nodes of the individual networks. Rows 4 and 5 use the edge weights of the individual networks. Rows 6 and 7 combine individual nodes and edges. The two first columns focus on a single data modality. Columns 3 to 6 refer to data integration.

FIG. 5 shows a comparison of classification results using graph-based models described herein to multiple classification algorithms applied to the raw features. Models are ranked according to their prediction performance. The lower the area in the coloured lines, the better. (a) shows the average rank of each model across datasets (prostate, brain, lung) and data types (RNAseq or histopathology). (b) shows the average rank of each model across datasets for the combined (i.e. concatenated) data types. For each analysis, the best graph approach is presented. Since the method based on IN's nodes is not using any graph structure information, only the approaches based on IN's edges, and IN's nodes-and-edges are considered as graph-approaches. (a) shows that for single data types, of the six analyses conducted, the graph-base approaches outperformed the other models in four cases (i.e. ⅔ of cases).(b) shows for the combined data types that the graph based approaches performed best in the lung and brain use cases.

FIG. 6 shows the results of a LIMMA analysis on the top 50 most differentially co-expressed edges between groups (see Example 4). Genes with absolute t-statistic<1.5 are shown in white. In the prostate use case (a), edges/nodes are red if they have higher coefficients in the Gleason pattern 3 group (blue for pattern 4). This shows that the most connected genes included MAP7, which is prognostic for survival in patients with stage II colon cancer. In the Brain use case (b), edges/nodes are red if they have higher coefficients in Brain lower grade glioma (blue in glioblastoma multiforme). In the Lung use case (c), edges/nodes are red if they have higher coefficients in Lung adenocarcinoma (blue in lung squamous cell carcinoma). Thicker edges represent higher log-fold changes.

FIG. 7 illustrates the principles of person-to-person networks (PPN) and individual networks (IN). (a) Multi-modality fusion from Person-to-Person networks. Nodes are individuals and edges show how close 2 individuals are. (b) Individual network. Nodes and/or edges are individual-specific.

FIG. 8 shows the results of assessment of classification performance (macro F1 scores (%)) obtained for classifications using PPNs from INs, using multiple data transformation to compute similarity between graphs (see Example 2). Top: INs from RNAseq data. Bottom: INs from histopathology data. No graph information is used when inferring the similarity matrix using the Spearman correlation, the Euclidean distance or the Gaussian kernel. Only graph information are studied when similarities are computed from individual graphs built with the Node Product or the LIONESS algorithm. Both raw data and graph information are investigated when a combination of the similarity matrices obtained with Spearman correlation and the Node Product of, with Spearman correlation and Lioness.

FIG. 9 shows the results of assessment of classification performance (macro F1 scores (%)) obtained for classifications using PPNs from INs with fusion of graphs from different data modalities at different levels (see Example 3). A: Graphs constructed using the Node product methodology. B: graphs constructed using the Lioness approach. Only one data type is used for values RNAseq and Histopathology. Data types are combined at early stage via the concatenation of the two databases (early), at intermediate stage via average of the similarity matrices (average) or SNF procedure (SNF) and at late stage via the majority vote (late). Note that the late integration is only performed when the combination of raw data and graph data are used so that the majority vote is applied on more than 2 outcomes.

FIG. 10 shows results of a gene set enrichment analysis on INs (see Example 4). The figures display the top 10 enriched gene sets from the largest component obtained from LIMMA analysis with features selected as described in Example 1 for a prostate cancer subject set (top) and lung cancer subject set (bottom). No enriched gene set was detected in the Brain cancer use case (see Example 1). The size of a pathway represents the number of genes in this pathway after removing genes not present in the largest component. For prostate cancer the most significantly enriched set is the Chandran metastasis pathway. Metastasis is the most adverse outcome in cancer.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Personalized screening prior to therapy paves the way toward improving diagnostic accuracy and treatment outcomes in multiple diseases including cancer. However, most approaches are limited to a single type of data and do not consider the interactions between features, leaving aside the complementary insights that multimodality and systems biology can provide. The inventors demonstrate the use of graph theory, and in particular subject-specific networks, for this purpose. Networks are powerful tools that consider interactions between pairs of features and therefore can better make use of all of the information available in a dataset, compared to considering only the values of said features individually.

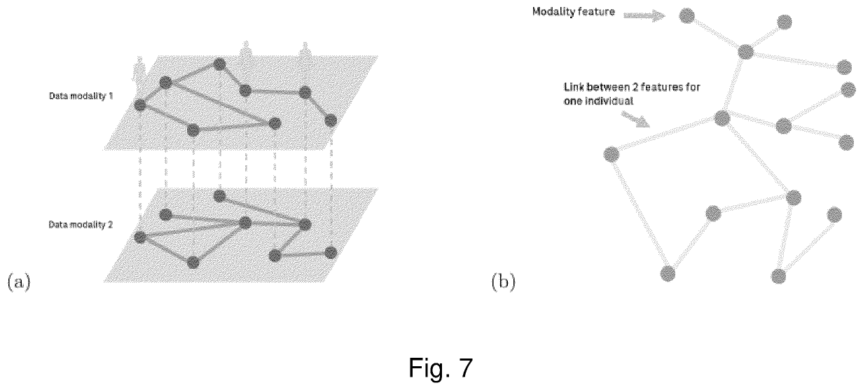

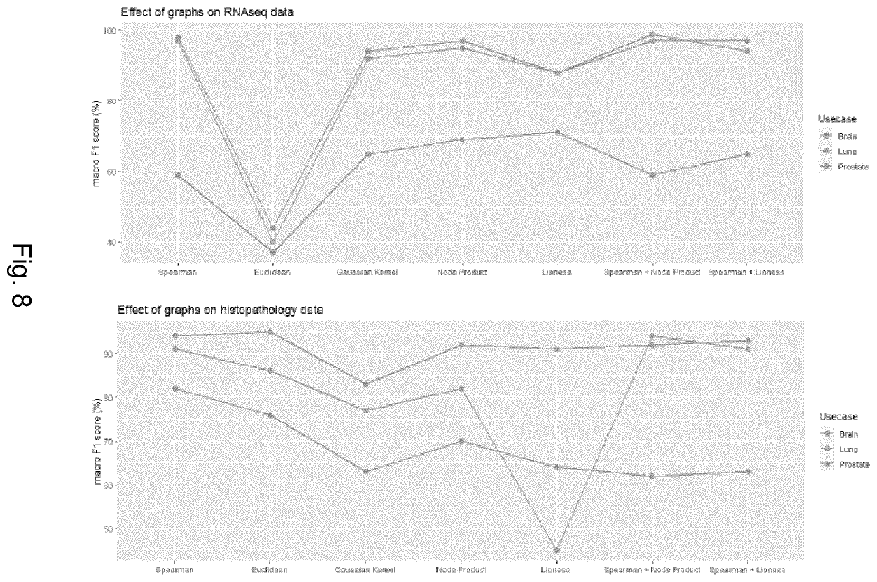

The present disclosure relates at least in part to methods that make use of subject-specific networks (also referred to as “individual networks”, INs). INs are networks where the values and/or presence of nodes and/or edges are individual specific. A “network” or “graph” is a data structure G comprising a set of nodes V, and a set of edges E between nodes (G=(V,E)). In the context of the present disclosure, edges in a graph can be described by an adjacency matrix A, with coefficients A(i,j) indicating the presence (e.g. when A(i,j) is 0 or 1 when no edge is present or when an edge is present, respectively) or weight of a relationship (edge) between nodes i and j. When edges are associated with weights, the network be referred to as a weighted network. In the context of the present disclosure, the networks are typically weighted networks, with edges weights indicating a relationship between the two nodes that it connects. A relationship between nodes (i.e. an edge) is quantified using any metric known in the art to quantify relationships between variables, such as e.g. node product (the product of the -optionally normalised—values of the two nodes connected by the edge, where the values of the nodes is typically equal to the value of the variables corresponding to the nodes connected by the edge), correlation, mutual information or other or co-variation metric (such as e.g. context likelihood of relatedness, described in Akhand et al. 2015) between the values of the two variables corresponding to the nodes connected by the edge, or path/message related metrics (such as e.g. weights obtained using the PANDA, Passing Attributes between Networks for Data Assimilation, algorithm as described in Glass et al. 2013), and metrics derived from any of the above such as gain or loss in the values of these metrics when adding or removing an individual from a cohort of subjects for which an aggregate network is obtained. In an individual network, all nodes and edges values are associated with a specific individual. However, depending on how the edges are obtained, they may have been determined using data about a cohort of individuals (such as e.g. when the edges represent gain or loss in edges weights obtained when comparing a network obtained for a cohort that comprises the individual vs the same cohort without the individual), or using the individual data alone (e.g. when edges are determined using the node product method). An individual network is illustrated schematically on FIG. 7(b). For each individual, nodes are variables (also referred to as “biological factors”) (e.g., genes), and edges show the link (also referred to as “relationship”) between these variables for that individual. Most prior art graph analyses methods for complex diseases aggregate information across a whole cohort, failing to detect individual characteristics. The inventors postulated that exploiting individual-specific interactions rather than population-level systems will help capture the heterogeneity between individuals and enhance the identification of new biomarkers for precision medicine. This hypothesis underlines the choice to use individual networks (INs). Since INs represent individual relations between variables, the inventors postulated that they can readily be used for precision medicine. Individual networks can be inferred via multiple approaches. For example, variables values (e.g., gene expression) for individuals can be superimposed to a reference network obtained from external knowledge (e.g., protein interactions), as described in Menche et al. 2017. With such an approach, only node values will differ between individuals and not the graph topology. Another option is Linear Interpolation to Obtain Network Estimates for Single Samples (LIONESS, described in Kuijier et al. 2019). LIONESS computes edge weights from the difference in edge weights for a network constructed using all the samples and a network reconstructed using all but the sample of interest. Another option is the single sample networks based on the Pearson correlation (ssPCC) algorithm, described in Liu et al. 2016. These individual networks are derived from the perturbation of the Pearson correlation caused by the addition of a particular individual to a given group of samples. Both LIONESS and ssPCC use a reference panel/group of samples. Alternatively, an edge weight can be computed without a reference panel by adding Z-scores of log-transformed values of the two associated nodes, as described in Koh et al. 2019, or by using repeated measurements per variable per individual (and e.g. computing the correlation between pairs of variables across repeated measurements).

The present disclosure relates in parts to methods that combine supervised data integration and individual networks. In particular, the present disclosure provides methods comprising: obtaining an individual network for a plurality of individuals comprising a disease subject and a reference set of subjects, computing a similarity (i.e. determining the value of a similarity metric) between the plurality of individual networks obtained, and using a machine learning model to predict a diagnosis or prognosis for the disease subject, wherein the machine learning model has been trained to predict a diagnosis or prognosis using as input the similarity between the plurality of individual networks of the reference set of subjects. To the best of the inventors' knowledge, such an approach had never been attempted or evaluated. The present inventors show that such an approach can predict disease subtype and severity from patient data, using data from two or more modalities and combining the modalities at various stages of the method. Indeed, the individual networks can be obtained for multiple modalities together (i.e. INs comprising nodes that are associated with different modalities), or individually for respective modalities (single modality INs). The former results in multi-modality INs and may be referred to as “early fusion”. When single modality INs are used, they can be combined by computing similarity between the plurality of INs for the same modality (single modality similarities), then combining the single modality similarities into a multi-modality similarity. This may be referred to as “intermediate fusion”. This is illustrated on FIG. 7(a), where individual-to individual networks are used to represent similarities between individual for each modality, and these similarities can be mapped to each other at the individual level and combined across multiple modalities. Alternatively, single modality INs may be used to compute single modality similarities, which may in turn be used to obtain single modality predictions using respective machine learning models trained on similarity inputs from a single modality. These single modality predictions can then be combined into a multi-modality prediction (late fusion). Note that all combinations of the above approaches for various subsets of a plurality of data modalities are also possible and envisaged. For example, a method may comprise obtaining one or more single modality INs for one or more respective modalities, obtaining one or more multi-modality INs each combining data for a plurality of modalities (early fusion), obtaining corresponding single modality and multi-modality similarities, combining at least some of the obtained similarities (e.g. one or more subsets of the single modality similarities) into a multi-modality similarity (intermediate fusion), obtaining predictions for each of the resulting similarities, and combining the predictions (if the similarities comprise multiple similarities, i.e. not all similarities were combined at the preceding step; late fusion).

The term “data modality” refers to data that has been obtained (i.e. recorded or measured) about a subject and that is from a specific source (also referred to as “type”). For example, a data modality may be data about gene expression (transcriptomics data, data about the presence and/or level of one or more transcripts) obtained from one or more samples from the subject, data about protein expression (proteomics data, data about the presence and/or level of one or more proteins) obtained from one or more samples from the subject, metabolomics data (data about the concentration of one or more metabolites) obtained from one or more samples from a subject, genomics data (data about the presence and/or characteristics of one or more genomic features, such as somatic mutations (of any kind including single base substitutions, indels and rearrangements), polymorphisms, epigenetic marks, copy number variations, etc.) obtained from one or more samples from the subject, demographic data (e.g. data about a subjects, age, gender, ethnicity, etc.) about the subject, histopathology data obtained from one or more samples from the subject, medical imaging data obtained from the subject (e.g. MRI, endoscopy, x-ray, ultrasound, CT scans, etc.), clinical data (e.g. comorbidities, treatment history, exposure factors, etc.) about the subject, microbiome data obtained from one or more samples from the subject (e.g. presence and/or abundance of one or more microbial taxa in one or more samples from the subject). Each of such data may be obtained from a sample, may be recorded in one or more databases or may have been previously obtained from a sample and recorded in one or more databased from which it can be obtained for the purpose of performing the methods described herein. For each of one or more data modalities, a data set comprises biological factors, which are values for a plurality of biological features, or data from which such values can be obtained. Individual data sets may have been obtained separately for individual samples, or may combine data from multiple samples from the same subject. Individual datasets are typically obtained using the same measurement technology. For example, a single gene expression dataset may comprise gene expression data (e.g. RNA sequencing data) obtained from one or more samples from the subject. A single histopathology dataset may comprise whole slide images or parts thereof obtained from one or more samples from the subject. The images may all be from the same sample (e.g. same tissue slide), and may correspond to different areas of the sample or different measurement channels (e.g. corresponding to different markers). Biological factors may be values for one or more features that are measured about the subject or one or more samples from the subject. These values may have been subject to one or more of normalisation, standardisation, filtering, etc. prior to use. For example, in the context of transcriptomics the features may be expression of respective genes, and the biological factors may be expression levels for the respective genes. The same principles can be applied to any data modality that measures the presence or level of any biological molecule or entity (transcript, metabolite, protein, microbial taxa, mutation, etc.) For clinical data the features may be exposure to one or more risk factors, presence or number of comorbidities, presence of a particular treatment history, etc., and the biological factors may be values that indicate whether the one or more risk factors are present or to which extent they are presence, the number or presence of comorbidities, the presence or absence of a particular treatment history, etc. Biological factors may be values for one or more features that are derived from values of features that are measured about the subject or one or more samples from the subject. For example, biological factors may be values for one or more features that are derived from measured values by applying data one or more data reduction approaches (e.g. values of one or more principal components obtained from a set of measured values by applying principal component analysis) and/or one or more machine learning models, where the biological factors are latent variables of such machine learning models. Thus, biological factors may be values of one latent variables of a machine learning model taking as input a set of measured values about the subject or one or more samples from the subject. The set of measured values may be e.g. pixel values for one or more images (e.g. medical images or histopathology images). In such cases, the machine learning model may be any machine learning model that has been trained for image processing. The machine learning model may have been at least partially trained (e.g. trained from scratch or fine-tuned) using training data comprising images of the same type as the images to be analysed (e.g. histopathology images, when histopathology images are used to generate biological features). The machine learning model may be a model that has been trained for generic image object recognition task. The machine learning model may be a model that has been trained (fully or by fine-tuning of a pretrained model) for a specific classification or regression task associated with the types of images of the image to be analysed. For example, in the context of histopathology images of tumour samples, the machine learning model may be a machine learning model that has been trained to classify histopathology images between a plurality of cancer types or subtypes. The task may be different from the prediction task that is the final object of the methods described herein (i.e. the task may be different from the diagnosis or prognosis prediction performed as part of the methods described herein). In the context of the present disclosure, edges are typically undirected.

Gene expression data refers to data about the expression level of an expression product of a gene. Within the context of the present disclosure, expression levels of genes of interest are preferably determined at the nucleic acid level, and in particular at the mRNA level. Thus, reference to a “gene expression level” may refer to a transcriptomic expression metric. A plurality of gene expression levels may be referred to as an expression profile or collectively a gene expression dataset. By “gene expression data” is meant a set of data relating to the level of expression of a plurality of genes in an individual. The determination of gene expression levels may involve determining the amount of mRNA for a particular gene or set of genes in a sample. Methods for doing this are well known to the skilled person. Gene expression levels may be determined in a sample using any conventional method, for example using nucleic acid microarrays, using nucleic acid synthesis methods (such as quantitative PCR, qPCR, also referred to as qRT-PCR), using molecular counting assays, or using RNA sequencing (including bulk RNA sequencing and single cell RNA sequencing). For example, gene expression levels may be determined using a NanoString nCounter Analysis system (see, e.g., U.S. Pat. No. 7,473,767). When using single cell RNAseq, pseudo-bulk RNA expression data can be obtained for the whole or one or more parts of the sample (such as e.g. one or more combined RNA expression levels can be obtained based on expression levels for respective pluralities of cells in respective specific population of cells).

For example, a gene expression dataset obtained from single cell RNA sequencing may comprise a first set of gene expression levels for cells in a first population (e.g. normal cells), and a second set of gene expression levels for cells in a second population (e.g. cancer cells). Expression levels for each gene represented in the first set and each gene represented in the second set (optionally after feature selection) may be used as values of nodes according to methods of the disclosure. The same concept is extendable to any number of populations of cells.

The disclosure relates to methods that include a step of determining a similarity between individual networks. The terms “similarity” and “distance” are used interchangeably to refer to metrics that quantify how similar or dissimilar two networks are from each other. These comprise similarity metrics, which quantify how similar two networks are from each other, and distance metrics, which quantify how dissimilar two networks are from each other. A similarity metric can be converted into a distance metric and vice versa using functions such as s(x,y)=u/(1+d(x,y)) where s is a similarity metric, d is a distance metric, x and y are two networks to be compared, and u is an upper bound, or d(x,y)=s(x,x)+s(y,y)−2*s(x,y) for kernel-based similarity measures. A distance/similarity metric may be computed separately for the edges of a network and for the nodes of a network. A distance/similarity metric computed solely based on the nodes of two networks does not reflect the similarity between the networks as it does not take edges into account. However, a distance/similarity metric computed solely based on the edges of a pair of networks does reflect the similarity between the networks. Further, a distance/similarity metric computed based on both the edges of a pair of networks and the nodes of the pair of network does reflect the similarity between the networks. Such a distance/similarity metric may be obtained by combining (e.g. summing or averaging) a distance/similarity metric obtained based on the edges of the pair of networks and a distance/similarity metric obtained based on the nodes of the pair of networks.

An edge distance/similarity metric may be selected from: Euclidian distance, Jaccard distance, edge difference distance, DeltaCon, spectral distances, graphlet-based measures, Hamming distance, Shortest path kernel, k-step random walk kernel, graph diffusion distance and Portrait Divergence. Euclidian distance, Jaccard distance, edge difference distance and DeltaCon distance are applicable when there is node correspondence between the two networks being compared. This is the case when all nodes are labelled and the same nodes are present in the two networks being compared. In embodiments, the similarity between INs is calculated using the edge difference distance metric. This distance takes two adjacency matrices (matrix A comprising coefficients A(I,i) equal to the weights of the edges between each pair of nodes i,j), and computes the Frobenius norm of their differences. This was found to strike a good balance between being informative (leading to good prediction performance) and being computationally efficient. Spectral distances, graphlet-based measures, Portrait Divergence, Hamming distance, Shortest path kernel, k-step random walk kernel and graph diffusion distance are applicable even without knowledge of the correspondence between nodes, but compare the global structure of networks and therefore are not suited to compare fully connected networks with nodes correspondence.

A node distance/similarity metric may be selected from: Euclidian distance, affinity matrix, Gaussian kernel, cosine similarity and spearman correlation. The spearman correlation coefficient was found to strike a good balance between being informative (leading to good prediction performance) and being computationally efficient. When classifying subjects based on nodes alone, the use of the Spearman correlation coefficient led to the highest classification accuracy on test data. An affinity matrix distance may be determined from a distance matrix, such as e.g. an Euclidian distance between each pair of nodes (corresponding nodes in the two individuals to be compared). A Gaussian kernel distance can be calculated as

$$k ( x , y ) = exp ( \frac{- {‖ {v}_{x} - {v}_{y} ‖}^{2}}{{σ}^{2}} )$$

where vₓ and v_{y} are the vectors of node values for individuals x and y, ∥vₓ−v_{y}∥ is the Euclidean distance between these vectors and σ² is a parameter corresponding to the bandwidth of the kernel. This value may be set empirically, for example σ=1000 was found to be suitable. The cosine similarity between to vectors vₓ and v_{y} is calculated as

$$Sc ( {v}_{x} , {v}_{y} ) = \frac{{\sum }_{i = 1}^{n} {v}_{xi} {v}_{yi}}{\sqrt{{\sum }_{i = 1}^{n} {v}_{xi}^{2} {\sum }_{i = 1}^{n} {v}_{yi}^{2}}}$$

where n is the size of vectors vₓ and v_{y}.

A network distance/similarity metric may be selected from: an edge distance/similarity metric, and the combination of an edge distance/similarity metric and a node distance/similarity metric. Multiple similarity metrics may be combined for example by summing or averaging.

Methods of the disclosure may comprise one or more feature selection steps applied to INs. Feature selection refers to the process of selecting nodes and/or edges of an IN for further analysis. Feature selection may be data driven, and/or based on prior knowledge. For example, nodes (i.e. variables) that are known to be more likely to be informative for a particular purpose may be included in an IN to be analysed (or used for training of a machine learning model), whereas nodes (i.e. variables) that are known to be less likely to be informative may be excluded. The same principles apply to edges. For omics data, edges may be selected for example based on pathway information from one or more databases, for example by including edges between genes or proteins that are known to interact with each other and excluding edges between genes or proteins that are not known to interact with each other. Data driven feature selection refers to the process of selecting edges and/or nodes purely based on the data available for an individual or cohort of individuals. Feature selection based on a cohort of individuals leads to the same set of nodes and edges for all individuals. Feature selection based on single individuals can lead to a different set of nodes and/or edges being selected for each individual in a cohort. An example of feature selection based on single individuals includes selecting nodes that are significantly different in the individual compared to a reference or control population (e.g. a cohort of individuals considered normal). An example of feature selection based on a cohort of individuals comprises selecting nodes that have a variability across the cohort that satisfies one or more predetermined criteria. For example, the top x nodes that have the highest standard deviation across individuals in the cohort may be selected. The value of x may be selected for example based on computational requirements (as lower values reduce the computational load required to implement the method) and/or based on prediction performance evaluated on a test dataset. For example, x may be the value that, when used to select nodes for inclusion in INs for the purpose of the present method, results in predictions with the highest accuracy (e.g. highest macro F1 score) on a test cohort of samples. Alternatively, the nodes that have a standard deviation above a threshold y may be selected, where the threshold y may be chosen as described above for the value x. Instead or in addition to this, feature selection based on a cohort of individuals may comprise obtaining a first network for a first subset of the cohort and a second network for a second subset of the cohort, and selecting the top x edges that have the largest difference between the two networks, or the edges that have a difference between the two networks above a threshold y and/or the edges that have a statistically significant difference between the two networks. The values of thresholds x and y can be selected as described above. The first and second networks may be obtained by calculating a correlation coefficient (e.g. Pearson correlation coefficient) between each pair of nodes across individuals in the first and second subsets, respectively. Any methods known in the art for obtaining a network that aggregates data for a set of subjects may be used. The first and second subsets of individuals may have different known diagnosis or prognosis. These may match to the diagnosis or prognosis labels that the method aims to predict. Feature selection using a cohort of individuals may in such cases be performed on training data comprising subjects with known diagnosis/prognosis.

Methods of the present disclosure comprise using a machine learning model to obtain a prediction for a subject. The machine learning model is model trained using supervised learning. This uses training data (also referred to as “reference data”) comprising biological features for a plurality of individuals, and ground truth labels indicating the value of the diagnosis or prognosis to be predicted. The machine learning model may be a classification model or a regression model. The machine learning model may be any machine learning model that can take as input a vector of similarity between an individual and a set of reference individuals, and produce an output indicative of the value of a diagnosis or prognosis to be predicted. This may be in the form of a class label, probability of belonging to one or more classes, or a value that is a predicted estimate of the diagnosis or prognosis value to be predicted. The machine learning model may be a support vector machine (SVM), a naïve bayes classifier, a k-nearest neighbour classifier, a classification or regression tree, or a neural network. Classification models may be particularly suitable to prediction of disease subtypes, or discrete severity scores or categories corresponding to ranges of severity scores. Regression models may be suitable for the prediction of continuous values such as continuous severity scores, survivability metrics (e.g. OS, DFS, PFS, as explained below), likelihood of survival, etc. The machine learning model may be an SVM. SVMs are suitable for both classification and regression tasks. A SVM algorithm identifies a hyperplane in an N-dimensional space (where N is the number of features associated with each instance to be classified) that best classifies the instances between predetermined classes. This is performed by identifying instances from different classes that are most similar to each other. A kernel-based SVM operates directly on a transformation of an original set of feature vector that represents the similarity between pairs of instances represented by these original features. Many machine learning algorithms can be expressed in terms of dot products between vectors to be compared, and any such machine learning algorithm can be used in the present methods. This includes e.g. SVM, logistic regression, perceptrons, etc.

The disclosure relates in parts to methods of providing a disease diagnosis or prognosis for a subject. A diagnosis may be the identification of a disease subtype. Disease subtyping refers to the identification of homogeneous groups of patients, i.e. patients that share molecular, histological and/or clinical characteristics. For example, many cancers comprise multiple subtypes that are associated with different aetiologies (e.g. tissues of origin), different histological features (e.g. lung adenocarcinoma vs lung squamous cell carcinoma), different molecular features (e.g. driver mutations, gene expression patterns, etc), and/or different phenotypic features (e.g. hormone dependent vs non-hormone dependent cancers).

A prognosis may be the identification of a disease severity (e.g. grade, stage or severity score), or likely outcome (e.g. a prediction of whether a subject has a good or bad/poor prognosis, belongs to a group of subjects that has good prognosis or a group of subjects that has bad prognosis). Disease severity may be assessed using a disease severity score, grade or stage. These are typically disease specific and assessed using a plurality of criteria. For example, the Gleason score is used to assess severity of prostate cancer, and is assessed based on histopathology data. It is obtained by detecting the appearance of cancerous cells in a biopsy: a biopsy comprising cells that look similar to normal prostate tissue is assigned Grade 1, a biopsy comprising mostly cells that look similar to normal cells is assigned Grade 2, and a biopsy comprising tumour cells is assigned one of Grades 3 to 5 (depending on how abnormal the cells look). The score is associated with prognosis in that the likely growth of the cancer correlates with the score (Grade 1 cancers are likely to grow very slowly, Grade 5 cancers are likely to grow very quickly).

Whether a prognosis is considered good or poor may vary between disease contexts (e.g. cancer type, stage of the disease, etc). In general terms a good prognosis is one where the overall survival (OS), disease free survival (DFS) and/or progression-free survival (PFS) is longer than that of a comparative group or value, such as e.g. the average forthat stage and cancertype, orthe average fora comparative group of subjects (e.g. a group of subjects that clusters separately). A prognosis may be considered poor if OS, DFS and/or PFS is lowerthan that of a comparative group or value, such as e.g. the average for that stage and type of cancer, or the average for a comparative group of cancers. Thus, in general terms, a “good prognosis” is one where survival (OS, DFS and/or PFS) and/or disease stage of an individual patient can be favourably compared to what is expected in a population of patients within a comparable disease setting. Similarly, a “poor prognosis” is one where survival (OS, DFS and/or PFS) of an individual patient is lower (or disease stage worse) than what is expected in a population of patients within a comparable disease setting.

A “sample” as used herein may be a cell or tissue sample (e.g. a biopsy), or an extract from which biological material can be obtained for analysis, such as transcriptome analysis (whole transcriptome sequencing, or targeted (also referred to as “panel”) sequencing), genomic analysis (e.g. genomic sequencing), proteomic analysis, histopathology analysis. For example, the sample may be a tumour sample or a blood sample. In the context of histopathology the sample may be a tissue sample, such as a tumour sample. In the context of cancer prognosis or diagnosis a sample may be a tumour sample or a biological fluid sample, for example comprising circulating tumour DNA ortumour cells. The sample may be one which has been freshly obtained from a subject or may be one which has been processed and/or stored priorto making a determination (e.g. frozen, fixed or subjected to one or more purification, enrichment or extractions steps). In particular, the sample may be a cell or tissue culture sample that has been derived from a tumour. As such, a sample as described herein may refer to any type of sample comprising biological material from which biological features may be determined. Further, the sample may be transported ad/or stored, and collection may take place at a location remote from the biological data acquisition (e.g. sequencing) location, and/or the computer-implemented method steps may take place at a location remote from the sample collection location and/or remote from the biological data acquisition (e.g. sequencing) location (e.g. the computer-implemented method steps may be performed by means of a networked computer, such as by means of a “cloud” provider). A “tumour sample” refers to a sample that contains tumour cells or genetic material derived therefrom. The tumour sample may be a cell or tissue sample (e.g. a biopsy) obtained directly from a tumour.

As used herein “treatment” and “therapy” refer to reducing, alleviating or eliminating one or more symptoms of the disease which is being treated, relative to the symptoms prior to treatment.

A subject or individual according to the present disclosure is preferably a mammalian (including a human or a model animal such as mouse, rat, etc.), preferably a human. The terms “patient”, “subject” and “individual” are used interchangeably. The patient may be a patient who has been diagnosed as having or being likely to have a disease. Thus, providing a diagnosis may comprise confirming a diagnosis of a disease, or providing a diagnosis of a subtype (including molecular subtypes, histopathological subtypes, phenotypic subtype, therapy response groups, severity groups, or any other distinction of groups of patients or disease, etc.) of a disease that the patient has been diagnosed as having.

The systems and methods described herein may be implemented in a computer system, in addition to the structural components and user interactions described. As used herein, the term “computer system” includes the hardware, software and data storage devices for embodying a system or carrying out a method according to the above-described embodiments. For example, a computer system may comprise a processing unit such as a central processing unit (CPU) and/or graphics processing unit (GPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Methods of Characterising a Subject

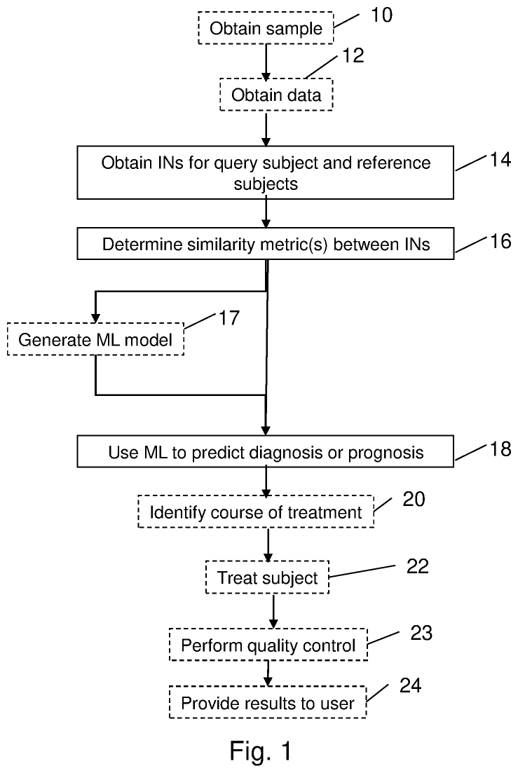

FIG. 1 is a flow diagram showing, in schematic form, a method of characterising a disease subject according to the disclosure. At optional step 10, one or more samples is/are obtained from a subject. At optional step 12, the samples are analysed to obtain a plurality of biological factors for each of one or more data modalities. This may comprise e.g. obtaining gene expression data (i.e. transcriptomic data) from a sample previously obtained from the subject, for example using RNA sequencing. This may comprise e.g. obtaining a histopathology image from a sample previously obtained from the subject. Other data modalities and combinations thereof are possible and explicitly envisaged, including e.g. demographic data about the subject (e.g. age, gender, ethnicity), clinical data about the subject (e.g. comorbidities, exposures such as e.g. smoking history), medical imaging data (including histopathology, MRI, x-ray, etc.), microbiome data about the subject (e.g. presence and/or amounts of one or more microbiological populations, e.g. microbial taxa, in a sample previously obtained from the subject), metabolomics data (e.g. amounts of one or more metabolites and/or values of one or more metabolic fluxes in a sample previously obtained from a subject), genomic data (e.g. presence of one or more genomic features such as mutations (including single base substitutions, multiple base substitutions, insertions, deletions and rearrangements), copy number variations and/or chromosomal instabilities), proteomic data (e.g. presence or amounts of one or more proteins in a sample previously obtained from the subject—including targeted and untargeted assays such as e.g. measurements of specific cytokines in samples from subjects), physiological data about the subject (e.g. from wearable devices), etc. The data preferably includes at least one omics modality (e.g. transcriptomics, proteomics, metabolomics, genomics) and/or one or more imaging modality (e.g. histopathology images). The value of a biological factor may be a value that has previously been subject to one or more transformations such as normalisation, standardisation, log transformation, etc. For example, node values (i.e. values of biological factors assigned to nodes in an individual graph) may be normalised using a min-max normalisation algorithm. Normalisation may be performed for each individual graph separately. The plurality of biological factors may comprise at least some biological factors related to a disease. The biological factors on which the similarity metrics (e.g. similarity matrices) are based may comprise at least one gene or protein expression and histopathology reading.

The biological factors may comprise latent variables of a trained machine learning model applied to image data, optionally wherein the image data is histopathology data. The trained machine learning model may be a machine learning model, optionally a neural network, that has been trained in a supervised manner to take as input histopathology data and provide as output a disease type label. The trained machine learning model may be a computer vision model. The trained machine learning model may be a deep neural network, such as a ResNet. The machine learning model may have been trained using a plurality of histopathology images from samples of a plurality of different cancer types. The machine learning model may have been trained to predict a cancer type for a histopathology image. The plurality of different cancer type may include a cancer type of the patient for which a prognosis or diagnosis is being predicted. The plurality of different cancer types may include at least 10 or at least 20 different cancer types.

At step 14, one or more individual networks are generated for each of a plurality of individuals using the biological data obtained at step 12, each individual network comprising a plurality of nodes and edges between pairs of the nodes, wherein each node is indicative of a biological factor in the biological data for an individual, and each edge is indicative of a relationship between a pair of biological factors corresponding to the nodes that the edge connects for the respective individual. The one or more individual networks may be referred to as individual graphs. They may comprise or consist of a set of nodes and a set of edges between the nodes. Each node may correlate to one of the biological factors. Edges between nodes may correlate to relationships between the biological factors for the respective individual.

Generating, for each of the plurality of individuals, one or more individual networks may comprise selecting a plurality of nodes each indicative of a biological factor in the biological data for an individual for inclusion in each respective individual network, wherein the selection is performed separately for each individual or collectively for the plurality of individuals, optionally wherein selecting a plurality of nodes comprises selecting a plurality of biological factors that are different between an individual and a reference set of individuals or selecting a plurality of nodes that have a variability across the plurality of individuals that satisfies one or more predetermined criteria. Generating, for each of the plurality of individuals, one or more individual networks may comprise selecting a plurality of edges indicative of a relationship between a pair of biological factors corresponding to the nodes that the edge connects for the respective individual, wherein the selection is performed separately for each individual or collectively for the plurality of individuals. Selecting a plurality of edges may comprise selecting a plurality of edges that are different between an individual and a reference set of individuals or selecting a plurality of edges that are different between a plurality of subsets of the plurality of individuals. Selecting a plurality of edges may comprise selecting a plurality of edges that are different between a first subset of the plurality of individuals and a second subset of the plurality of individuals. The first and second subsets may be subsets associated with a first and second prognosis or diagnosis to be predicted. Similarly, the plurality of subsets may be subsets associated with a plurality of different prognosis or diagnosis to be predicted (e.g. different cancer types, different cancer severity groups, etc). Edges that are different between different subsets of individuals may be edges that have a difference between networks obtained for the respective subsets that is above a predetermined threshold. Nodes/edges that are different may refer to differences above a predetermined threshold or to top x most different nodes/edges, where x is a predetermined value. Selecting a plurality of nodes that have a variability across the plurality of individuals that satisfies one or more predetermined criteria may comprise selecting nodes that have a variability (e.g. standard deviation) above a predetermined threshold, or selecting the top x most variable nodes where x is a predetermined value.

At step 16, the value of one or more similarity metrics between one or more individual networks generated for the patient and one or more individual networks generated for other individuals in the plurality of individuals is/are determined. Determining the value of one or more similarity metrics may comprise calculating one or more similarity matrices representing the similarity between the individual graphs. The one or more similarity metrics may comprise, for each of a plurality of pairs of individual networks, a similarity between edges in the individual networks, a similarity between edges in the individual networks and a similarity between nodes in the individual network, or a similarity combining a similarity between edges in the individual networks and a similarity between nodes in the individual network. A similarity between edges in individual networks may be referred to as a similarity between the individual networks (graphs). A similarity between nodes in individual networks may be referred to as a similarity between the biological factors independent of the graphs (individual networks). The one or more similarity metrics may comprise, for each of a plurality of pairs of individual networks: a similarity between nodes in the individual networks obtained as a Spearman correlation coefficient, an affinity matrix, or a Gaussian kernel using a distance metric between vectors corresponding to the nodes in the respective individuals, or a similarity combining a similarity between edges in the individual networks and a similarity between nodes in the individual network obtained as a Spearman correlation coefficient, an affinity matrix, or a Gaussian kernel using a distance metric between vectors corresponding to the nodes in the respective individuals. A similarity metric obtained as a Gaussian kernel using a distance metric between vectors corresponding to the nodes in the respective individuals may be calculated as

$$( x , y ) = exp ( \frac{- {‖ {v}_{x} - {v}_{y} ‖}^{2}}{{σ}^{2}} )$$

where vₓ and v_{y} are the vectors of node values for individuals x and y, ∥vₓ−v_{y}∥ is the Euclidean distance between these vectors and σ² is a parameter corresponding to the bandwidth of the kernel.

The one or more similarity metrics between one or more individual networks may comprise one or more similarity metrics obtained by combining, for a pair of individuals, a plurality of similarity metrics each derived from a pair of individual networks for the respective individuals obtained from data comprising values of biological factors obtained using a different set of one or more data modalities. For example, the one or more similarity metrics between individuals i and j may comprise a similarity metric obtained by combining (i) a similarity metric between an individual network obtained for individual i and an individual network obtained for individual j using a first data modality (e.g. gene or protein expression); and (ii) a similarity metric between an individual network obtained for individual i and an individual network obtained for individual j using a second data modality (e.g. histopathology). Any number of similarity metrics obtained from INs derived from any number of data modalities may be used. Combining similarity metrics may be performed using summing or averaging. Such a process may be referred to as intermediate fusion. In embodiments, all similarity metrics may have been obtained by combining, for a pair of individuals, a plurality of similarity metrics each derived from a pair of individual networks for the respective individuals obtained from data comprising values of biological factors obtained using a different set of one or more data modalities.

At step 18, a diagnosis or prognosis is predicted for the patient using a machine learning model configured to predict a diagnosis or prognosis of the disease in the patient, wherein the machine learning model has been trained to take as input the values of one or more similarity metrics between individual networks and produces as output a diagnosis or prognosis. Predicting a diagnosis or prognosis for the patient using the machine learning model may comprise predicting a diagnosis or prognosis of the disease in the patient based on the machine learning model and based on biological factors obtained from the patient. In other words, the trained machine learning model may be used together with similarity metrics (e.g. similarity matrices) obtained from biological factors for the patient, to predict a diagnosis or prognosis.

The machine learning model may comprise a plurality of machine learning models, each machine learning model configured to predict a diagnosis or prognosis of the disease in the patient, wherein each machine learning model has been trained to take as input the values of a respective subset of the one or more similarity metrics between individual networks and produce as output a diagnosis or prognosis, wherein the respective subsets of similarity metrics are derived from individual networks that are generated from values of biological factors obtained using respective data modalities, and wherein providing a diagnosis or prognosis for the patient comprises combining the outputs of the plurality of machine learning models. Such an approach may be referred to as late fusion. Combining the outputs of the plurality of machine learning models may be performed by averaging (e.g. when the outputs are continuous) or by majority voting (e.g. when the outputs are a classification).

The results of step 18 may be used to select patients for a particular course of therapy based on any prognostic or diagnostic feature as described above, to select patients for a clinical trial based on features of samples from said patients that identify the patient as likely responsive to a therapy, or to provide a prognosis or diagnosis that is associated with the predicted feature (e.g. prognosis associated with a predicted disease subtype). Thus, at step 18, the subject may be classified as having a good or poor prognosis. Instead or in addition to this, the subject may be selected for participation in a clinical trial. Instead or in addition to this, the subject may be classified at step 18 as being likely to respond or unlikely to respond to a particular course of treatment. At optional step 20, a particular course of treatment (which may comprise one or more different individual therapies) may be identified based on the results of step 18. For example, a subject that has been identified at step 18 as unlikely to respond to the particular course of therapy may be identified as likely to benefit from a therapy that is different from the particular course of therapy. Alternatively, a subject that has been identified at step 18 as likely to respond to the particular course of therapy may be identified as likely to benefit from a therapy that includes the particular course of therapy. As another example, a subject that has been identified at step 18 as having poor prognosis may be identified as likely to benefit from a more aggressive course of treatment than a subject that has been identified at step 18 as having good prognosis. As another example, a subject that has been identified at step 18 as having a first type of disease may be identified as likely to benefit from a therapy that is indicated for this first subtype of disease. At optional step 22, the subject may be treated with the therapy identified at step 20.

At optional step 24, results of any one or more of steps 12 to 20 may be provided to a user.

The subject is preferably a human patient. The subject may be a subject who has been diagnosed as having cancer. Thus, the disease that is being characterised may be cancer. The cancer may be ovarian cancer, breast cancer, endometrial cancer (uterus/womb cancer), kidney cancer (renal cell), lung cancer (small cell, non-small cell and mesothelioma), brain cancer (gliomas, astrocytomas, glioblastomas), melanoma, merkel cell carcinoma, clear cell renal cell carcinoma (ccRCC), lymphoma, gastrointestinal cancer (e.g. colorectal cancer), small bowel cancers (duodenal and jejunal), leukemia, pancreatic cancer, hepatobiliary tumours, liver cancer (e.g. hepatocellular carcinoma), germ cell cancers, prostate cancer, head and neck cancers, bladder cancer, thyroid cancer, oesophagal cancer, melanoma (e.g. uveal melanoma), cutaneous squamous cell carcinoma and sarcomas. For example, the cancer may be head and neck squamous cell carcinoma (HNSCC), hepatocellular carcinoma (HCC), colorectal cancer (CRC), different types of lung cancer (LC), clear cell renal cell carcinoma (ccRCC), prostate cancer (PC), breast cancer (BC), bladder urothelial carcinoma (BUC), esophageal squamous-cell carcinoma (ESCC), uveal melanoma (UV) and cutaneous squamous cell carcinoma (cSCC). The cancer may be brain cancer, lung cancer or prostate cancer.

The prognostic or diagnostic feature predicted at step 18 may be a cancer diagnosis or prognosis. The prognostic or diagnostic feature predicted at step 18 may be a disease subtype (e.g. a cancer type) or a disease severity (e.g. cancer severity or grade). A cancer severity or grade may be a score calculated using any severity metric known in the art. A cancer severity metric predicted at step 18 may be a Gleason score. A disease severity may be a risk score, such as e.g. a risk of metastasis/recurrence. A cancer subtype may be any cancer subtype known in the art. For example, in the context of lung cancer a cancer subtype may be selected from lung adenocarcinoma (luad) and lung squamous cell carcinoma (lusc). Thus, step 18 may comprise classifying the subject (who has or is suspected of having lung cancer) as having lusc or luad (i.e. classifying the subject between a first class comprising subjects with lusc and a second class comprising subjects with luad). As another example, in the context of brain cancer, a cancer subtype may be selected from lower grade glioma (Igg) and glioblastoma multiforme (gbm). Thus, step 18 may comprise classifying the subject (who has or is suspected of having brain cancer) as having Igg or gbm (i.e. classifying the subject between a first class comprising subjects with Igg and a second class comprising subjects with gbm).

The method may further comprise an optional step 17 of generating the machine learning model configured to predict a diagnosis or prognosis of the disease in patients. The machine learning model may have been trained with the one or more similarity matrices and biological factors. Thus, the method may comprise training the machine learning model using the biological data comprising values for a plurality of biological factors for the plurality of individuals, optionally not including the patient for whom a prediction is being made. The machine learning model may have been trained or may be trained as part of the method using training data comprising values for a plurality of biological factors for the plurality of individuals, and a known prognosis or diagnosis for all individuals except for the patient for whom a prediction is being made. Generating the model may comprise: obtaining, for each of a plurality of training individuals biological data comprising values for a plurality of biological factors for the individual, and a diagnosis or prognosis label associated with the individual; generating, for each of the plurality of individuals, one or more individual networks, each individual network comprising a plurality of nodes and edges between pairs of the nodes, wherein each node is indicative of a biological factor in the biological data for an individual, and each edge is indicative of a relationship between a pair of biological factors corresponding to the nodes that the edge connects for the respective individual; determining the value of one or more similarity metrics between one or more individual networks generated for the patient and one or more individual networks generated for other individuals in the plurality of individuals; and generating a machine learning model configured to predict a diagnosis or prognosis of the disease in a patient, wherein the machine learning model takes as input the values of the one or more similarity metrics between individual networks and produces as output a diagnosis or prognosis. The method may be performed in the context of performing quality control for biological data about a patient with a disease. Thus. also described herein are methods comprising characterising the disease in the patient as described in relation to steps 10-18 using biological data about the patient comprising values for a plurality of subsets of biological factors obtained using respective different data modalities;

Characterising the disease in the patient as described in relation to steps 10-18 using biological data about the patient comprising only values for a first subset of biological factors; and performing quality control of the data at optional step 23, by comparing the predicted diagnosis or prognosis obtained using the plurality of subsets of biological factors and the first subset of biological factors, wherein a predicted diagnosis or prognosis being different for the first subset of biological factors compared to the plurality of subsets of biological factors is indicative of poor quality of the biological data comprising the first subset of biological factors.

### Systems

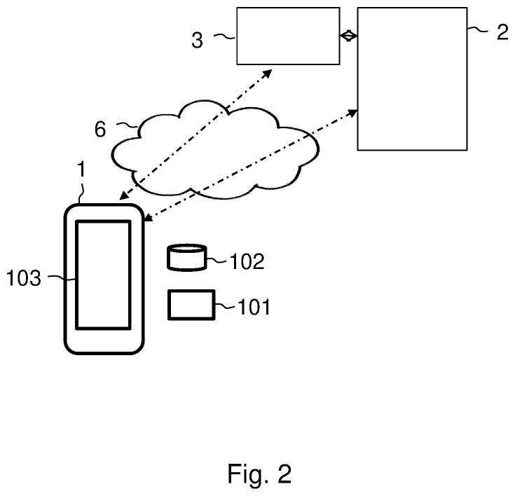

FIG. 2 shows an embodiment of a system for characterising a subject and/or for providing a prognosis, diagnosis or treatment recommendation, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network, to biological data acquisition means 3, such as a e.g. a sequencing machine, microscope, mass spectrometer, etc., and/or to one or more databases 2 storing biological data (values of a plurality of biological factors). The one or more databases 2 may further store one or more of: one or more machine learning algorithms, training data, parameters (such as e.g. parameters of machine learning model, feature selection algorithm, IN calculation method etc.), clinical and/or sample related information, etc. The computing device may be a smartphone, tablet, personal computer or other computing device. The computing device is configured to implement a method for characterising a disease subject, as described herein. In alternative embodiments, the computing device 1 is configured to communicate with a remote computing device (not shown), which is itself configured to implement a method of characterising a disease subject, as described herein. In such cases, the remote computing device may also be configured to send the result of the method to the computing device. Further, the various steps of the methods described herein may be split between the computing device 1 and the remote computing device. Communication between the computing device 1 and the remote computing device may be through a wired or wireless connection, and may occur over a local or public network 6 such as e.g. over the public internet. The biological data acquisition means may be in wired connection with the computing device 1, or may be able to communicate through a wireless connection, such as e.g. through WiFi and/or over the public internet, as illustrated. The connection between the computing device 1 and the biological data acquisition means 3 may be direct or indirect (such as e.g. through a remote computer). The biological data acquisition means 3 are configured to acquire biological data comprising values of a plurality of biological factors from sample previously obtained from a subject. The biological data acquisition means 3 may comprise a gene expression data acquisition means, such as a next generation sequencer, and/or a histopathology data acquisition means, such as a microscope. The following is presented by way of example and is not to be construed as a limitation to the scope of the claims.

### EXAMPLES

Personalised cancer screening before therapy paves the way toward improving diagnostic accuracy and treatment outcomes. Most approaches are limited to a single data type and do not consider interactions between features, leaving aside the complementary insights that multi-modality and systems biology can provide. In these examples, the inventors demonstrate the use of graph theory for data integration via individual networks where nodes and edges are individual-specific. They showcase the consequences of early, intermediate, and late graph-based fusion of RNAseq data and histopathology whole-slide images for predicting cancer subtypes and severity. The methodology demonstrated is as follows: 1) create individual networks; 2) compute the similarity between individuals from these graphs; 3) train a model on the similarity matrices; 4) evaluate the performance using the macro F1 score. Pros and cons of elements of the pipeline are evaluated on publicly available real-life datasets. The inventors show that graph-based methods can increase performance over methods that do not study interactions. Additionally, merging multiple data sources often improves classification compared to models based on single data, especially through intermediate fusion. The proposed workflow is demonstrated in the context of cancer but can be adapted to other disease contexts to accelerate and enhance personalized healthcare.

### Example 1—Prediction of Outcomes Via Individual Graphs

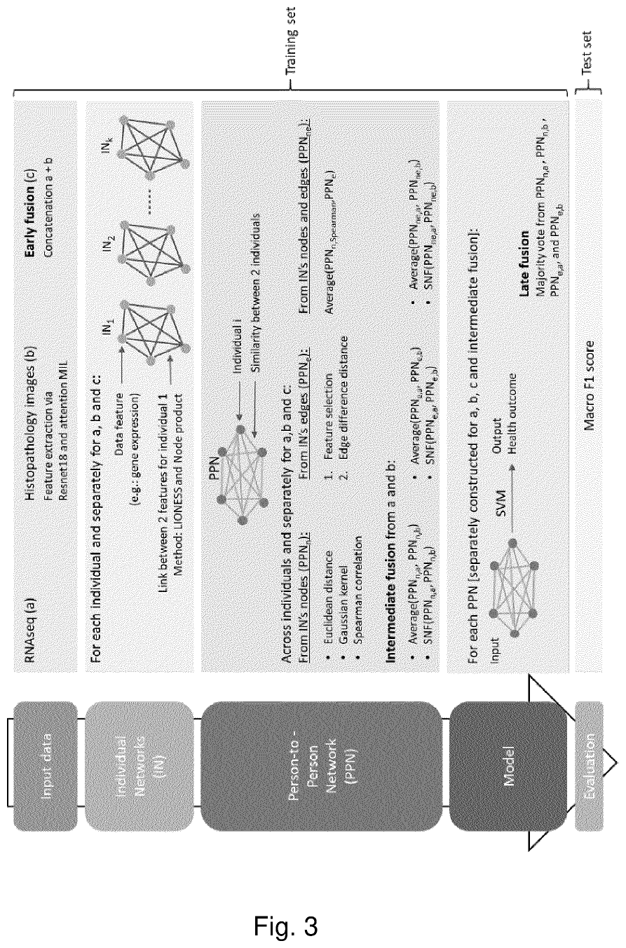

In this example, the inventors describe a newly developed multi-step workflow (see FIG. 3) to predict outcomes via individual graphs. First, a network is constructed for each individual: nodes and/or edges are specific to an individual. From these individual networks, we compute a similarity matrix that we call a Person-to-Person Network (PPN): nodes are individuals and edges represent how similar individuals are. Various levels of information from the individual graph are used to build the Person-to-Person network: nodes, edges, or nodes and edges. The Person-to-Person network becomes the input of the machine learning model. In other words, we considered the similarities to a reference panel as variables. Then, the outcome is predicted from these similarities to a reference set.

### Methods

Data. Results on the impact of using individual graphs or not are based upon publicly available real-life data generated by the TCGA Research Network [49](https://www.cancer.gov/tcga). For each patient, two types of data were used: images (in particular, histopathology Whole Slide Images) and genomic data (in particular, gene expression data obtained by RNA sequencing). For images, features were extracted as described below. For RNAseq data, features were the read counts for each gene. These examples focus on three use cases: prostate cancer severity using the Gleason score, Brain low-grade gliomas (Igg) versus Glioblastoma multiforme (gbm) differentiation, and lung adenocarcinoma (luad) versus lung squamous cell carcinoma (lusc) differentiation. For each use case, we analysed two data modalities: RNAseq data and histopathology Whole Slide Images (WSI). Prostate cancer is the most common cancer in men, and prostate cancer stages are commonly described according to the Gleason Score, which helps evaluate the prognosis (Egevad et al. 2002). This score is derived from the appearance of cancerous cells that can correspond to 5 patterns (normal to tumour cells). Grade 1 cells do not differ from normal prostate tissue; grade 5 corresponds to tumour cells. Thus, cancers with a higher Gleason score are more severe. Physicians determine the Gleason score by looking at biopsy samples and assigning one grade to the predominant pattern (primary Gleason score). Usually, a second Gleason grade is given to the second most predominant pattern, and the two grades are added to set the secondary Gleason score. These examples focus on the primary Gleason score and specifically on patterns 3 and 4. In this work, the inventors examine if the newly developed workflow can highlight the differences between these two patterns. The database contains 297 individuals in the training set (130 patterns 3, 167 patterns 4) and 71 in the testing set (34 patterns 3, 37 patterns 4).

Brain low-grade gliomas (Igg) are cancerous brain tumours. They arise from the support cells in the brain. Glioblastoma multiforme (gbm) is an aggressive cancer in the brain or spinal cord. Studies have already identified variations between these two tumours, such as gender-specific molecular differences. Here, the inventors study if INs and combining RNAseq and histopathology data can help identify these two brain tumours. The training set contains 344 individuals (282 Igg, 62 gbm), and the testing set 156 individuals (122 Igg, 34 gbm).

Lung adenocarcinoma (luad) and lung squamous cell carcinoma (lusc) are among the most common lung cancer subtypes and are both considered non-small cell lung cancer (NSCLC). They have different biological signatures, but these variations in their biological mechanisms remain to be disentangled even though recent studies have made progress [6]. The training data contains 603 patients (232 luad, 371 lusc), and the testing data has 140 patients (50 luad, and 90 lusc).

Gene expression data processing. Publicly available gene expression data from TCGA (portal.gdc.cancer.gov) have been used. Only samples from primary tumor sites were selected. We downloaded Gene expression data in fragment per kilobase million (FPKM) with 56,602 Ensemble gene identifiers. We normalized the FPKM data into TPM (Transcripts per million) using following equation:

$$TPM = \frac{{FPKM}_{i}}{\sum {FPKM}_{i}} * 1 {0}^{6} .$$

Only protein-coding genes were included, using Ensembl annotations. Genes with zero expression values were excluded.

Feature extraction—histopathology. Feature extraction is performed on the histopathology Whole Slide Images (WSI). We considered the full TCGA dataset with 30 cancertypes, including the types to classify in the 3 use cases described above (i.e., brain, lung, and prostate cancers), but excluding the individuals in the testing sets. A pretrained neural network model is applied to differentiate between the cancer types. In particular, we use Resnet18 (He et al. 2015) and attention MIL (Ilse et al. 2018), trained for 100 epochs on all TCGA slides, sampling 128 random tiles per slide every epoch. An imageNet pretrained ReNet18 classifier was pretrained then used to create embeddings which were used by an Attention MIL model trained to provide the classification based on the embeddings. 512 features contained in layer N−1 are selected as new variables. Each feature is a vector of length the number of individuals and contains discriminative information for cancer type. We assumed that the difference in cancer types would provide relevant information for differentiating the groups in our 3 use cases. Thus, a table consisting of individuals in rows and neural network features in columns is used as the input data for histopathology information.

Similarity between individuals—single data sources. Three types of approaches to computing similarity between individuals were tested, explained in more detail below. In the first type of approaches, a similarity is obtained based solely on node values. This is a baseline in the sense that no network information is used. In the second type of approaches, a similarity is obtained solely based on interactions between variables (edges). In the third type of approaches, a similarity is obtained based on both the nodes (raw data) values and the edges values.

Single data source—level of individual nodes. We created baselines where only the node weights of the individual networks are used, i.e., only the raw feature values. We will refer to them as node level approaches. This can also be referred to as “raw data” approaches. This approach can be considered not relying on individual network structures as the interactions between features are not used in the model. Three ways of obtaining a Person to-Person network PPNₙ with these individual graphs were tested. PPNₙ(x, y) indicates how similar individuals x and y are. The first option was to use the Euclidean distance between each pair of individuals' features and to compute an affinity matrix that represents the neighbourhood graph of the individuals. This was performed using the function affinity Matrix in package SNFtools (Wang et al. 2021). This function takes three arguments: a distance matrix (in this case obtained with the Euclidean distance), a parameter K, and σ. K is the number of neighbours, where affinities outside of the neighbourhood are set to zero, and affinities inside are normalised. σ is a hyperparameter for the scaled exponential similarity kernel used to conduct the actual affinity calculation. These parameters were chosen empirically (K=20, σ=0.05). A variation was to apply a Gaussian kernel. This was performed using the function gausskernel from the package KLRS (Ferwerda et al. 2017). Given two vectors vₓ and v_{y} of descriptive variables for individuals x and y, the Gaussian kernel is defined as

$$k ( x , y ) = exp ( \frac{- {‖ {v}_{x} - {v}_{y} ‖}^{2}}{{σ}^{2}} )$$

where ∥vₓ−v_{y}∥ is the Euclidean distance and σ² (here, σ=1000) is the bandwidth of the kernel. The third option was to compute the Spearman correlation between each pair of patients. This was performed using the function rcorr from the package Hmisc (Harrell 2021).

Single data source—level of individual edges. A second type of Person-to-Person network PPNₑ was computed to measure the impact of considering the interactions between variables. Specifically, we built a network for each individual where nodes are variables and edges represent the link between these variables. Because individual networks can be very large when the number of variables per data source increases, we performed feature selection at the node and edge levels. It allowed us to focus on relevant signals, remove noise and decrease the computing time. Alternatively, this may be done only for the gene expression data and for the imaging data the number of variables can be controlled at the step of obtaining the features (e.g. based on the architecture of the model which dictates the number of latent variables, e.g. using regularisation). First, we selected data features (i.e., nodes) with the highest standard deviation across individuals in the training set (i.e. k most variably expressed genes). Then, we created condition-specific networks for each subgroup to predict. We calculated the difference between these condition-specific network adjacency matrices and selected edges with large absolute differences in their co-expression levels (calculated as node products, see below). We select the edges that have a difference in Pearson R correlation coefficient of at least I. To define the thresholds k and l, we performed a stratified 5-fold cross-validation within the training set and chose the parameters giving rise to the higher macro F1 score on average.

We derived individual edge weights based on two approaches. In the first one, which we called Node Product, we applied a minimum-maximum normalisation algorithm across all variable values to scale them between 0 and

$$1 : {i}^{′} = \frac{i - min ( i )}{max ( i ) - min ( i )}$$

with i and i′ a variable and normalised version thereof. Then, for an individual x, the weight of the edge between nodes i and j is e^{x}ᵢⱼ=i′^{x}*j′^{x} with i‘ and j’ the normalised versions of variables i and j. Since this method is computationally efficient, this is the one used to build INs for feature selection.

The second approach to create edge weights was the LIONESS algorithm (Kuijer et al. 2019). The general idea is to study the difference between a network constructed from all individuals and a network derived from all but one individual. If a difference appears, it must be due to the individual being left out. The LIONESS equation is the following: e^{x}ᵢⱼ=N(e^{α}ᵢⱼ−e^{α-x}ᵢⱼ)+e^{α-x}ᵢⱼ, where e^{α}ᵢⱼ is the weight of an edge between nodes i and j in a network modeled on all N samples and e^{α}ᵢⱼ is the weight of that edge in a network modeled on all samples except the sample of interest x. For each individual, we derived edge weights using the lionessR function (Kuijer, 2022). Notably, these edge weights are specific to the reference panel used to compute e^{α}ᵢⱼ.

Regardless of the method used, we reduced the INs obtained to the previously identified selection of edges. Finally, we applied the minimum-maximum scaling algorithm on INs so that the edge weights range between 0 and 1. In particular, we considered the minimum and maximum weights across all INs in the scaling so that the ordering of the weights remained the same between individuals. We used the similarities to individuals in the training sets as new variables for prediction.

Using as the predictor variable, how similar individuals are from a reference panel (here the training set) requires defining a measure of distance between individuals. Many methods have been developed to compare graphs. The specificities of our context limited the choice of distance. Indeed, the measure should be computed in a reasonable amount of time, even on large graphs, and should handle undirected and weighted networks. Since the same variables (e.g., same genes) are used for all the individuals, there is a node correspondence between the different INs (node 1 in INₓ₁ corresponds to node 1 in INₓ₂, where x and x2 are different individuals). This type of graph is called multiplex. We are in fact comparing multiple layers of the same graphs, that correspond to the different individuals. From the way we build the INs, in addition to having the same nodes, we have the same edges between individuals. Only the edge weights differed from one person to another. That implies that without additional filters, all the graph distances based on structural differences will not allow us to identify if some graphs are more similar than others. For example, the Euclidean, Jaccard, edge difference, or DeltaCon distances suited our context. We used the edge difference distance in this project because of its good computational properties. This distance takes two adjacency matrices and computes the Frobenius norm of their differences. We applied this distance to each pair of individual networks to obtain a matrix of similarity between individuals.

Inference of individual networks with the LIONESS algorithm on new patients. Computing how similar a new individual is to each individual in the training set is straightforward with the Node Product approach. However, with the LIONESS algorithm, the INs of the reference panel change when we consider a new individual. Also, the derivation of the IN for a new individual depends on this panel. Hence, to create INs for new individuals, we used all individuals from the training set and one new individual at a time. If we directly add all the new individuals to the ones from the train set (reference panel), we could considerably modify the relations between the INs of the training set. Adding only one individual at a time to the training set minimises the impact on the similarities between individuals from the train. To summarise, with the LIONESS algorithm, for each individual x from the test set, we created a new database containing the reference panel and x, and we constructed the INs for all the patients in this temporary database. From these new INs, we computed the similarity between x and each individual in the training set. Hence, the complexity of creating individual networks for individuals in the test set with the LIONESS algorithm motivates starting investigations using the Node Product approach.

Single data source—combination of individual nodes and individual edges. There is no reason to assume that individual node and edge information could not be complementary. Thus, we also investigated the combination of Person-to-Person networks built from individual nodes and individual edges. We built the Person-to-Person networks independently for the two approaches, and we averaged their corresponding adjacency matrices to merge them.

Data integration—early fusion. One option to integrate multiple database information was concatenating the original data (early fusion) and applying the pipeline as in the single data procedure. When using individual edges, or edges and nodes, we included the additional step: for each data source, the top variables are selected as described above. Multiple edge correlation thresholds (0.25, 0.5, and 0.75) are tested to reduce the INs further. Here, nodes can be variables from any of the original databases, and edges can therefore represent the association between any kind of variables.

Data integration—intermediate fusion. Fusion can also be performed at the PPN level (intermediate fusion). PPNs (similarity matrices) are obtained for each data source separately and merged to benefit from their potential complementary. Simple methods can be applied for this task, such as computing the average of the different PPNs (average similarity matrix). More advanced approaches include the Similarity Network Fusion (SNF) (Wang et al. 2014). SNF has proven efficient in combining multiple data such as mRNA expression, DNA methylation and microRNA expression data for cancer data. In this project, we tested both the average and the Similarity Network Fusion algorithms. Then, an SVM model was applied as described below.

Data integration—late fusion. The last alternative considered was late fusion, where data are merged after an independent investigation of each data source (a and b). With continuous outcomes, it can be computed by summation or averaging. Since we are performing classification, we used the majority vote approach. In most of our application settings, we considered two data modalities only, so a majority vote would not provide additional knowledge. Hence, we applied late fusion only on results obtained from Person-to-Person Networks derived from the combination of individual nodes and edges, where we consider prediction from four outcomes: PPN_{n,a}, PPN_{n,b}, PPN_{e,a}, and PPN_{e,b}. When two labels were predicted equally for an individual, the final label was randomly assigned to one of them.

Prediction and performance assessment. The similarities between individuals (Person-to-Person Networks, PPN(x,y)—similarity between INₓ and IN_{y}, where x and y are two individuals) were normalised with the following transformation:

$$PPN ( x , y ) = \frac{PPN ( x , y )}{\sqrt{PPN ( x , x ) PPN ( y , y )}} .$$

The normalised PPNs were used to train a Support vector machines (SVM) model using the kernlab package (Karatzoglou et al. 2004). Instead of working with the original sample representation in the original dimensional space, SVM classification methods operate directly on similarity matrices. We tested multiple options of parameter C (10^{k} for k=1, 2, 3, 4, 5), and we selected C giving rise to the best performance. Note that feature selection and hyperparameter tuning are performed on the same training set. Evaluation is then performed on an in dependent test set (as described above). We obtained the performance by comparison with the ground-truth labels for the test set data. As the groups are unbalanced, we used the macro F1 score to assess the performance, with Macro

$$F 1 score = \frac{1}{L} {\sum }_{l = 1}^{L} F 1 {score}_{i}$$

where I is the label index and L the number of labels.

### Results

A new data integration workflow is proposed as illustrated on FIG. 3. Three inputs are considered: the RNAseq modality, the histopathology images (inputs for intermediate and late fusion), and the concatenation of these two modalities (input for early fusion). An individual network is constructed for each input separately and for each individual of the training set. From these individual networks, a Person to-Person Network is built, where nodes are individuals and edges represent how close two individuals are. Either the nodes, the edges or the nodes and the edges from the individual graph are used to build the Person-to-Person network. The Person-to-Person network is used to train a support vector machine (SVM) model for each of three prediction tasks (classify individuals with prostate cancer as Gleason score 3 or 4, classify individuals with brain cancer as low-grade glioma or glioblastoma multiforme, and classify individuals with lung cancer as lung adenocarcinoma or lung squamous cell carcinoma). Then, the individual networks of the test set are computed. The similarities of the individuals from the test set to the individuals from the train set (reference set) are calculated to create the Person-to-Person network of the test set. The SVM model is applied to these similarities to a reference set, and the performance of the classification is determined using the macro F1 score. All analyses (i.e., the different types of modality integration and the different levels of information used to compute the Person-to-Person networks) are compared as explained in Examples 2 and 3 below.

### Example 2—Single Data-Source: The Effect of Exploiting Nodes, Edges, or Nodes and Edges in the Individual Graphs

### Methods

See Example 1.

Model comparison. We compared our graph-based approach to multiple classification methods applied to the raw features. Namely, we used a penalized logistic regression, a classification tree, a random forest, AdaBoost, and a naïve Bayes method. The algorithms were applied on each data type separately (RNAseq and histopathology features) and on the combined dataset (RNAseq and histopathology features concatenated). For each algorithm, we computed the associated macro F1score to show how our model and its variants compare to standard and state of-the-art classification methods. Note that these five models are only compared to the graph approaches based on IN's edges, and IN's nodes- and edges, as the approach based on IN's nodes is not using any graph structure in the process. Data were pre-processed as follows: constant variables and correlated variables (|r|>0.75) are removed. For the penalized logistic regression, we used the function cv.glmnet from the package glmnet (Friedman et al. 2010) with options alpha=1, lambda=NULL. For the random forest, we applied function randomForest from the package randomForest (Liaw and Wiener, 2002) with option ntree=500. For AdaBoost, we used the function boosting from the packageadabag (Alfaro et al. 2013) with option boos=TRUE, and mfina=50. For the classification tree, we applied the function rpart from the package rpart (Therneau and Atkinson, 2019) with the default options. For the naïve Bayes approach, we used the function naiveBayes from the package e1071 (Meyer et al. 2022) with the default options.

### Results

We compared the prediction performance obtained using different levels of information in the individual graphs (i.e. with and without use of the individual graphs). To create INs, feature selection is performed as described in Example 1. It gave rise to a selection of 700 genes (edge weight percentile threshold tedge=0.25) and 300 histopathology features (tedge=0.25) for the prostate use case. In the brain cancer dataset, 600 genes (tedge=0.5) and 300 image features (tedge=0.75) were retained. For the classification of the two lung cancers, 600 genes (tedge=0.5) and 500 histopathology features (tedge=0.75) were considered.

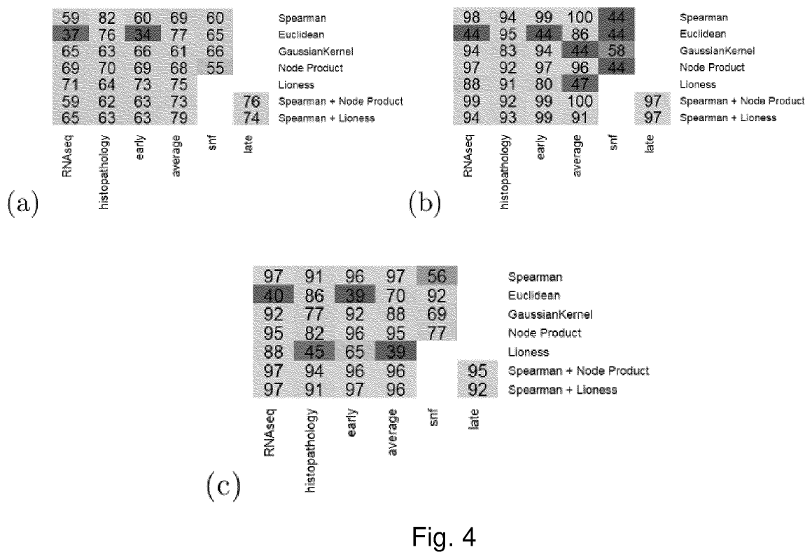

FIG. 4 shows the results of this comparison. The first two columns of each heatmap show the effects of using the nodes (rows 1 to 3), edges (rows 4 and 5), or nodes and edges (rows 6 and 7) of the individual network on each data modality. Additional visualisation is presented in FIG. 8. The Spearman correlation performed best in two-thirds of the scenarios among the three methodologies to build similarities at the node level (raw data only). It motivated the choice of the Spearman correlation for the combination of node level and edge level information.

We observed that using more than node information (i.e. using graphs) increased the macro F1 score for the prostate (max F1=0.71) and brain (max F1=0.99) use cases with RNAseq data, and for the lung use case (max F1=0.94) with histopathology data. Using individual edges or individual nodes led to an equal performance in the context of lung cancer (max F1=0.94) from RNAseq data. Pipelines based on node level information achieved higher prediction with histopathology data for the prostate (max F1=0.82) and brain classifications (max F1=0.94).

Among the two approaches to build similarity matrices via individual edges (i.e. using graphs), the Node Product performed better than the LIONESS algorithm in all situations except the prediction of prostate cancer severity using RNAseq data. However, when combining individual nodes and edges, the LIONESS method yielded higher results in half of the situations. In general, on single data, classification based on individual edge weights, with or without combination with individual node weights, was better or equal to predictions from individual nodes only (i.e. no individual graph structure) in two-thirds of the scenarios. In other words, the data show that classification based on individual graphs (with or without combination with raw data) is better or equal to raw data predictions in the majority of scenarios. Thus, these results highlight the high potential of individual graphs for disease subtyping.

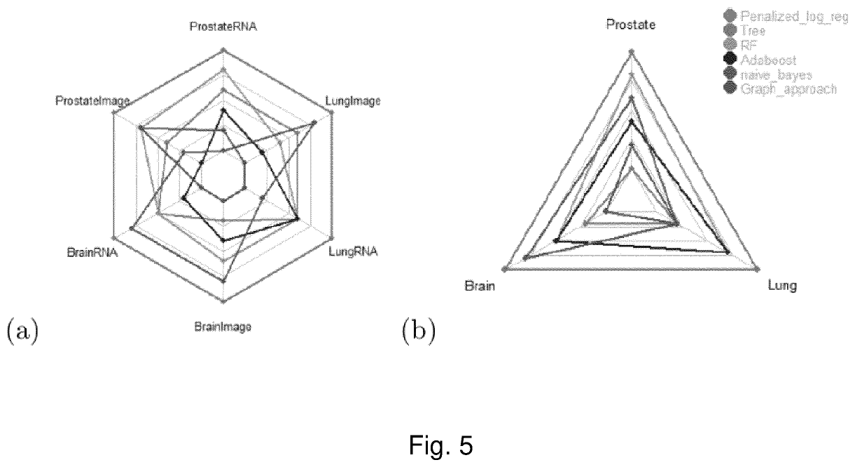

Moreover, we compared graph-based models to multiple classification algorithms applied to the raw features, separately for each data type: a penalized logistic regression, a classification tree, a random forest, AdaBoost, and a naive Bayes method. The models were ranked based on their macro F1 scores, with the best model ranked 1 and the worst model ranked 6. The results are presented in FIG. 5 (a), where a lower the area in the coloured lines indicates better performance. Among the six analyses conducted, the graph-based approaches outperformed the other models in four of them. Overall, the graph-based approaches performed the best, with an average rank of 1.75. Following closely, the adaboost algorithm achieved the second-best performance, with an average rank of 2.5. These results demonstrate the substantial potential of individual graphs for disease subtyping.

### Example 3—Multi-Data Integration: The Effect of Early, Intermediate and Late Integration

In this example, the inventors studied the impact on prediction performance of multi-modality integration using graphs. Specifically, they focused on three different fusions arising at different stages of the pipeline: early, intermediate or late.

### Methods

See Examples 1 and 2.

### Results

This analysis's first goal was to study whether it is possible to predict disease subtypes and severity from the patient data using graphs. The results showed that in brain cancer, the workflow achieved perfect predictions. We also obtained high performances in the lung cancer use case (macro F1 score=0.97). The severity of the prostate cancer was more cumbersome to detect, with a maximum macro F1 score of 0.82. The second goal of this study was to examine which modality yields the best prediction. The answer differs depending on the use case. The histopathology images were the most informative data in the prostate scenario, but the RNAseq data achieved better results for the lung and brain scenarios.

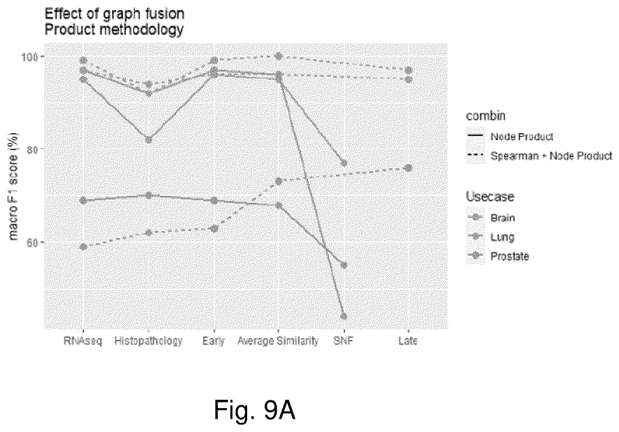

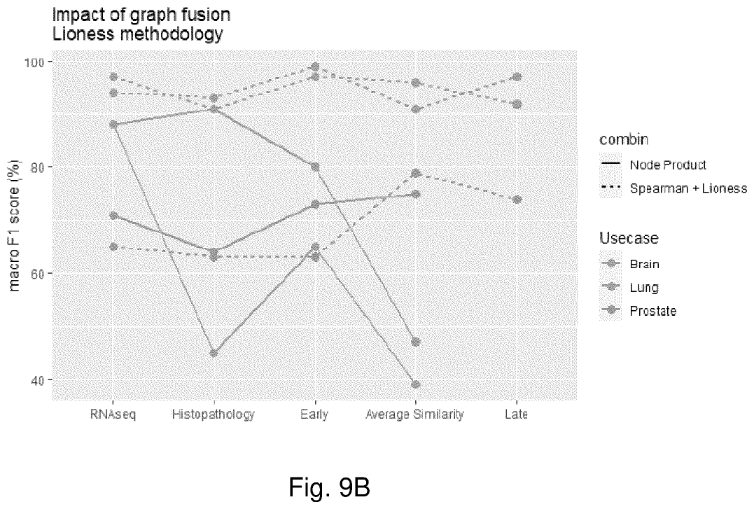

The third aim was to leverage the consequences of using INs and PPNs to combine database in formation at various steps. The impact of multi-modality integration using the edge weights of the individual graphs is shown in rows 4 and 5 of the heatmaps on FIG. 4. An alternative visualisation is presented in FIG. 9. With the Person-to-Person Networks derived from individual edge weights only (i.e., no node weights), the fusion of the two data sources provided better results for the prostate (max F1=0.75) and the lung (max F1=0.96) cancers. There was no difference between one modality or the fusion of two modalities for brain cancer (max F1=0.97). Hence, with Person-to-Person networks derived from individual edge weights only, there was a benefit of combining multi-modalities. In other words, with graphs only, the data shows a benefit of combining multiple modalities. There was no clear outperformer between the LIONESS and the Node Product methodologies.

When considering Person-to-Person networks computed from the combination of individual edge weights and individual node weights (FIG. 4, rows 6 and 7 of each heatmap), the fusion of the RNAseq and histopathology data produced improved predictions for the prostate (max F1=0.79) and the brain cancers (max F1=1). There was no observed difference for lung cancer since multiple pipelines produced the best macro F1 score of 0.97. Thus, with the combination of individual node and edge weights, we also observed that performance was improved in two-thirds of the situations with the fusion of the two data sources. In our examples, intermediate fusion via average similarity matrices (intermediate fusion) outperformed early or late fusion.

Finally, we compared results across all analyses: use of nodes and/or edges in the individual graphs, and use on one or two data modalities (entire heatmaps—FIG. 4). For prostate cancer, the two best results were obtained from the Spearman correlation on histopathology data and the average intermediate fusion of the two data types with the combination of node level and edge level information. The good performance observed with the histopathology images was expected since physicians determine the primary Gleason score by looking at biopsy samples. For the brain cancers, the best results were achieved via the average intermediate fusion of the two data modalities with Spearman correlation and via the intermediate fusion with the combination of node level and edge level information. Note that the brain classification was already perfect (macro F1 score=1) with node level information only, and there was, therefore, no possible improvement with individual edges. An ideal use case would require complementary data, each one bringing partial information. For lung cancers, the maximum macro F1 score was obtained from six different settings involving the Spearman correlation and the combination of Spearman correlation and approaches based on individual edges. The good performance observed with the combinations of individual nodes and edges for brain and lung cancers could be mainly due to the nodes only. Hence, no approach outperformed the others in all contexts, and no general rule could be derived.

In addition, we conducted further analyses to assess the added value of our graph-based models. Specifically, we compared these models to several classification algorithms applied to the raw features on the combined data types, where the features from the different data types were concatenated. The outcomes of these analyses are displayed in FIG. 5 (b). Among the three analyses conducted (brain, prostate and lung data), the graph-based approaches performed best in two of them. When considering the overall performance, both the graph-based approaches and personalized logistic regression exhibited the best results, with an average rank of 1.67. Then, the adaboost and naive Bayes algorithms achieved the next-best performance, with an average rank of 3.67. These findings further underscore that the graph-based models provide valuable insights and demonstrate their effectiveness in handling combined data types.

Together, the data show that graphs (with or without fusion) achieve very competitive performance, and are often beneficial even on a single data source. Therefore, the data shows that there is a benefit in considering individual networks for disease subtyping, because performance will be as good or better than using the raw data only, and even when the performance is not better the graph approach still provides additional opportunities such as e.g. interpretability, explainability and flexibility. Indeed, the approaches described herein can be easily extended to other types of data. Thus, the data shows the benefits of considering graph-based methods for supervised learning and in particular for multi-modality classification.

### Example 4—Interpretability

Graphs bring essential properties in terms of interpretability. For example, when nodes are genes, networks can easily be superimposed with external knowledge or compared to independent analysis results. In this example the inventors suggest associating the prediction with complementary approaches, such as LIMMA (Ritchie et al. 2015) and pathway analyses (Subramanian et al. 2005) to take advantage of the full potential of graphs.

### Methods

See Example 1.

LIMMA and gene set enrichment analysis on graphs. Originally, LIMMA is an analysis of gene expression data that uses linear models to simultaneously assess differential expressions between many targets. We applied the LIMMA analysis on RNAseq individual networks because genes are interpretable units of analysis. We selected the most differentially co-expressed pairs of genes and coloured edges depending on their values in the classes to predict. It identifies the edges whose weights differed significantly between the groups. In parallel, we also used LIMMA to test for significant differences in gene expression levels between groups. Specifically, we coloured nodes based on the t-statistic from the LIMMA analysis. In the resulting network, edges were therefore coloured based on whether they had higher or lower weights in patients from the different groups. Genes with absolute t-statistic<1.5 are shown in white, genes in red/blue have higher expression in patients from group a/b, respectively. Thicker edges represent higher log-fold changes. The resulting networks obtained by applying the LIMMA analysis on the features selected as described in Example 1 can further be investigated with a gene set enrichment analysis (Ben Guebila et al. 2022). We focused on the largest connected components. Since all the genes in that module are connected, they can indicate a broader biological mechanism responsible for the group difference. Forthe pathway analysis, we performed the LIMMA analysis on the features selected as described in Example 1, and we used the fgsea package (Sergushichev 2016) to perform gene set enrichment analysis with a minimum gene size of 10 and 5000 permutations. Two inputs were required: a ranked gene list and a list of gene sets to test for enrichment. For the former, we used the gene t-values of the genes in the largest component from the LIMMA analysis. It represents the gene statistical difference between the two groups compared. For the latter, we downloaded all ontology and curated Molecular Signature Database (MSigDB version 7) gene sets (Liberzon et al. 2015). We applied FDR cut-off of 0.05 for significant assessment.

### Results

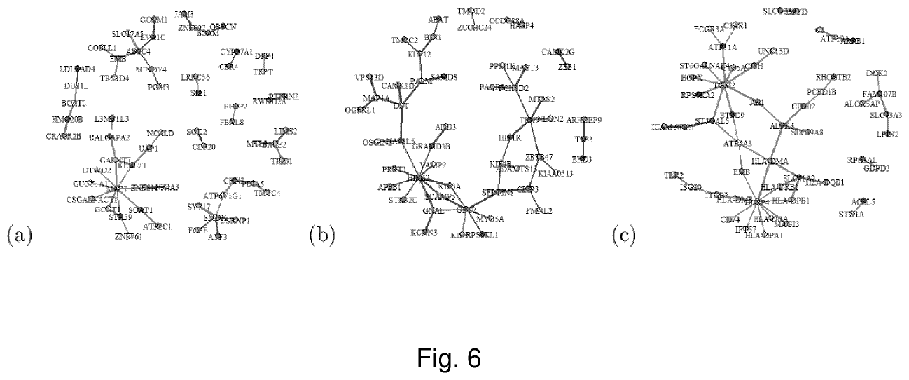

We applied a LIMMA analysis and a gene set enrichment analysis to the RNAseq data to illustrate the potential of individual networks to understand biological mechanisms. To detect which edge weights were significantly different between the classes (e.g., Gleason score 3 versus 4), the top 50 most differentially co-expressed edges were selected and coloured (See FIG. 6 a-b-c). Nodes with significant gene expression differences between groups were also identified based on the t-statistic from the LIMMA analysis. This visualisation gave a general overview of the organization of the most relevant gene pairs differentiating between groups while highlighting specific nodes and interesting modules. We can, for instance, investigate the most connected genes (more than 5 connected neighbours). The Gleason score classification pointed MAP7, which is prognostic for survival in patients with stage II colon cancer (Blum et al. 2008). In brain cancer prediction, GTP2 and HIPK2 were identified. GTP2 is linked to neurological disease, encephalopathy, and microcephaly (e.g. Hengel et al. 2018), and HIPK2 is associated with tumor progression, and malignant neoplasm (e.g. Garufi et al. 2019). In lung cancer differentiation, we detected TGM2 and DUSP4. A loss of DUSP4 is observed in EGFR-mutant tumours (Chitale et al. 2009). Hence, the graph approach helped target gene and gene pairs differentiating between the two investigated groups.

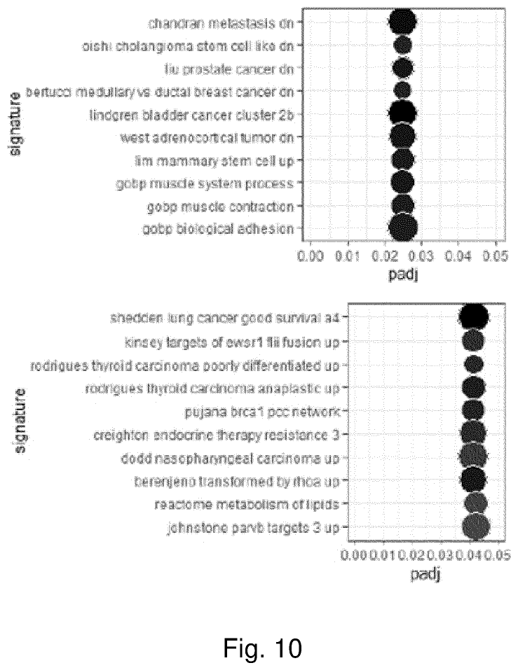

We performed gene set enrichment analysis to investigate the bio logical mechanisms associated with the difference between subtypes. It was based on the LIMMA analyses that include all the features identified as explained in Methods. Namely, 700 genes (edge weight per centile threshold tedge=0.25) are investigated for prostate cancer, 600 genes (tedge=0.5) for brain cancer, and 600 genes (tedge=0.5) for the lung cancer. 36 gene sets are enriched in the prostate cancer use case (See FIG. 10) and 10 gene sets in the lung cancer use case. No enriched pathway was detected for the two types of brain cancers. The most significant gene sets for the prostate analysis was the Chandran metastasis. In prostate cancer, metastasis represents the most adverse outcome, and it is assumed that genes associated with this pathway have a role in the biology of metastatic disease (Chandran et al. 2007). We also identified the Liu prostate cancer set (Liu et al. 2006) that is linked to a study showing that sex-determining region Y Box 4 is a transforming Oncogene in human prostate cancer cells. In the lung cancer scenario, the most significantly enriched gene set was Shedden lung cancer good survival a4 (Shedden et al. 2008), coming from the investigation of gene expression-based survival prediction in lung adenocarcinoma. Thus, these results highlighted the relevance of graphs in identifying biological processes involved in differentiating cancer subtypes.

### Example 3—Discussion and Conclusion

### Methods

See Examples 1 and 2.

### Results

Despite the increasing volume of human data, methods for data-modality integration are under studied. Commonly, late integration is performed manually and relies on prior knowledge of the disease studied. Moreover, biological mechanisms are often organized as complex systems. Allocating a network to each individual could model such interactions while accounting for individual specificities. Starting from these observations, we investigated the added value of individual graphs for cancer subtyping. We integrated data in the space of individuals rather than measurements (e.g., gene expressions) using networks of similarities between patients. First, we evaluated the benefits of transforming the input data into individual graphs on single data. We showed that considering the features as a connected system could improve the prediction performance. Second, we demonstrated that combining individual networks and multi-modality integration can yield better performance. Finally, as we illustrated with cancer data, one strength of graph-based approaches is the ability to visualise and provide insights into the causal factors accounting for the differences between the disease subtypes. Although we focus here on RNAseq and histopathology data, our framework applies to any multiplex data. In clinical studies, it offers opportunities to integrate various measurements such as demographic, microbiome, and metabolomics data. The use of person-to-person network (i.e. a matrix of similarity between individuals) as input to machine learning is advantageous because such a person-to-person network is completely independent of the original data, although it is derived from the original data. This means that any types of data can be combined to obtain such a PPN, then the PPN can be used for prediction as demonstrated.

Several choices have been made for the construction of the individual networks representing each patient. For example, in the lioness algorithm, our edge weights are based on Pearson correlation. Some studies have shown that bi-weights also yield nice results on RNAseq data. One of the lioness pitfalls is that the computation of the edge weights is based on a reference panel. Hence, this option is not as computationally efficient as node products because we need to recompute all the individual networks when adding a new individual. Also, this method considers the loss of information to create an IN since we look at the difference between a network obtained with a population and a network obtained with the population except for one individual. Another possibility could be to consider the gain of information instead to build such networks.

Another limit of our individual networks is that they all have the same structure: same nodes, same edges, and only the edge weights differ from one patient to another. This restricts our distance choice to evaluate how different two networks are. To tackle this problem, INs can be filtered One of the most simple and commonly used approaches to sparse networks is to set a threshold, for example, a quantile, and only consider edges that have a weight higher than the threshold. This quantile can be computed per individual (selection of the top edges per individual) or across individuals. If such an additional filter is used on individual networks to obtain different structures, measures such as spectral distances, graphlet-based measures, Portrait Divergence, or graph-kernel based measures can be tested. Focussing on relevant modules in each individual network is not only beneficial to be able to apply more advanced distances between graphs, but also enables to focus on predictive signals and remove noise. In our application, we consider node weights (from raw data) and edge weights (from lioness or Node Product) separately, and we combine raw data and graph data at the level of the similarity matrices, by computing the average. Another possibility is to use a distance between graphs which consider at the same time node weights and edge weights. Hence, the combination would be made at the level of the individual graphs instead of the level of the similarity matrices.

In this study, one data type in the cancer use cases was histopathology Whole Slide Images. This data was transformed beforehand to convert images to image features with continuous values. To achieve this transformation, we considered a dataset that included but was not restricted to the individuals of our use cases, and we applied a neural network model to predict the cancer type. From this model, we derived features that discriminate between cancer types. Even though it may seem more straightforward to apply a neural network for each use case and train on the label of interest (e.g., Gleason score), we observed that differentiating among cancer types yielded better results (data not shown). One possible explanation is that the image model trained purely on the groups to identify resulted in a not general enough embedding. Further, a more general model on cancer types contains more individuals, which may lead to a better embedding that recognizes essential features.

Future enhancements include a data integration strategy that takes advantage of graph specificities. In this work, we studied the impact of combining individual graphs and data integration, but we did not use the network characteristics in the integration itself. Data were integrated before the computation of individual graphs (early integration) or after the derivation of similarity matrices (intermediate and late integration). An alternative would be to combine the data within the process of creating individual networks. In FIG. 3, this would correspond to an intermediate integration occurring at the level of the second box (“Individual networks”). For instance, one could develop a method to select predictive features in individual networks obtained with a first database (e.g., RNAseq) from a second dataset (e.g., histopathology data). This approach could allow focusing on interpretable variables while including knowledge of an additional database. Second, it would sparse the individual networks, enabling more advanced graph distances to be used to compare individuals. Additionally, the approach can also accommodate features from a data modality that cannot easily be mapped to a node in an individual graph. In such cases, the relationship between individuals can be directly modelled in similarity matrices.

The proposed methodology is flexible and not specific to one machine learning model. The present examples used a Support Vector Machine model since this method operate directly on a similarity matrix. Another option is to create an embedding of the individual networks and apply another machine learning model, such as a random forest of neural networks. Notably, neural networks are often less interpretable and could have provided low performance because our sample sizes were small. The graph based approach proposed here advantageously enables multiple options in relation to the fusion stage, i.e. early, late or intermediate. By contrast, most prior art approaches are only able to accommodate early or late fusion. Additionally, our model considers as input data how similar individuals are from a reference data. That is already different from the most common models applied to RNAseq data which often consider up and down regulation of individual genes compared against a mean distribution. With our model, not only the weights of the machine learning model must be known to predict the group of a new individual, but also data of the reference panel must remain accessible.

The present examples further show that one advantage of protocols relying on graphs is their interpretability property. In this project, the inventors use a LIMMA analysis to visualize the genes and gene pairs having the biggest role in the differentiation of the group tested. In these examples, we are always differentiating between two groups only, but we may encounter situations where more than two classes need to be investigated. In that case, one can represent the LIMMA networks for each pair of groups, to highlight the genes and gene interactions responsible for each two-group difference. For example, if we were comparing 3 Gleason score groups (e.g. patterns 3, 4 and 5), we could represent the LIMMA networks for patterns 3-4, 3-5 and 4-5. It would show the genes responsible for the transition and hence for the evolution and severity of prostate cancer. Also, we decide to apply a gene set enrichment analysis to the largest component of the LIMMA network. Indeed, since all the genes in that module are connected, they can indicate a broader biological mechanism responsible for the group difference. Another possibility is to apply a pathway analysis developed specifically for networks, such as a Network neighbourhood search protocol (see e.g. Duroux et al. 2022) which considers the topology of the network using the shortest paths between the studied genes and a reference biological network.

Conclusion. Whereas research on disease subtyping has received significant attention recently, individual treatment decisions remain a cumbersome issue. Taking advantage of the complementarity of multiple data sources could help provide more precise subtypes. Ongoing research on multi-modality integration mainly considers one variable at a time, ignoring their interactions. Fusion based on individual graphs accounting for these interactions can bring additional information. In this study, we showcased the potential of graph theory. In particular, we underlined the advantages of this approach in the context of prostate, brain, and lung cancers subtyping and severity assessment. We observed that individual graphs could be beneficial even on single data sources, and we highlighted that intermediate integration was often among the best performers. Graph-based methods achieved competitive performance while bringing additional explainability properties. We identified biologically relevant genes, gene interactions, and pathways for different use cases. The presented workflow is flexible and can readily be applied to other data modalities. The results motivate more research on methodological developments of individual networks for precision medicine.

### REFERENCES

Esteban Alfaro, Matias Gamez, and Noelia Garcia. adabag: An R package for classification with boosting and bagging. Journal of Statistical Software, 54(2):1-35, 2013.
Jerome Friedman, Trevor Hastie, and Rob Tibshirani. Regularization paths for generalized linear models via coordinate descent. Journal of statistical software, 33(1):1, 2010.
Andy Liaw and Matthew Wiener. Classification and regression by randomforest. R News, 2(3):18-22, 2002
David Meyer, Evgenia Dimitriadou, Kurt Hornik, Andreas Weingessel, and Friedrich Leisch. e1071: Misc Functions of the Department of Statistics, Probability Theory Group (Formerly: E1071), TU Wien, 2022. R package version 1.7-11.
Terry Therneau and Beth Atkinson. rpart: Recursive Partitioning and Regression Trees, 2019. R package version 4.1-15.
Diane Duroux, Hector Climente-Gonzalez, Chloe-Agathe Azencott, Kristel Van Steen, Interpretable network-guided epistasis detection, GigaScience, Volume 11, 2022, giab093.
M. A. H. Akhand, R. N. Nandi, S. M. Amran and K. Murase, “Context likelihood of relatedness with maximal information coefficient for Gene Regulatory Network inference,” 2015 18th International Conference on Computer and Information Technology (ICCIT), Dhaka, Bangladesh, 2015, pp. 312-316
Glass K, Huttenhower C, Quackenbush J, Yuan G C. Passing messages between biological networks to refine predicted interactions. PLoS One. 2013 May 31; 8(5):e64832.
Ash et al. 2021. Jordan T Ash, Gregory Darnell, Daniel Munro, and Barbara E Engelhardt. Joint analysis of expression levels and histologi cal images identifies genes associated with tissue morphology. Nature communications, 12(1):1-12, 2021.
Schneider et al. 2022. Lucas Schneider, Sara Laiouar-Pedari, Sara Kuntz, Eva Krieghoff-Henning, Achim Hek ler, Jakob N Kather, Timo Gaiser, Stefan Fröhling, and Titus J Brinker. Integration of deep learning-based image analysis and ge nomic data in cancer pathology: A system atic review. European Journal of Cancer, 160:80-91, 2022.
Wang et al. 2014. Bo Wang, Aziz M Mezlini, Feyyaz Demir, Marc Fiume, Zhuowen Tu, Michael Brudno, Benjamin Haibe-Kains, and Anna Golden berg. Similarity network fusion for aggregat ing data types on a genomic scale. Nature methods, 11(3):333-337, 2014.
Speicher and Pfeifer, 2015. Nora K Speicher and Nico Pfeifer. Integrating different data types by regularized unsupervised multiple kernel learning with applica tion to cancersubtype discovery. Bioinfor matics, 31(12):i268-i275, 2015.
Glass et al. 2013. Glass K, Huttenhower C, Quackenbush J, Yuan G C. Passing messages between biological networks to refine predicted interactions. PLoS One. 2013 May 31; 8(5):e64832.
Kuijier et al. 2019. Marieke L Kuijjer, Ping-Han Hsieh, John Quackenbush, and Kimberly Glass. lionessr: single sample network inference in r. BMC cancer, 19(1):1-6, 2019.
Menche et al. 2017. Jörg Menche, Emre Guney, Amitabh Sharma, Patrick J Branigan, Matthew J Loza, Frédéric Baribaud, Radu Dobrin, and Albert László Barabási. Integrating personalized gene expression profiles into predictive disease-associated gene pools. NPJ systems biology and applications, 3(1):1-10, 2017.
Egevad et al. 2002. Lars Egevad, T Granfors, L Karlberg, A Bergh, and Per Stattin. Prognostic value of the gleason score in prostate cancer. BJU international, 89(6):538-542, 2002.
Ilse et al. 2018. Maximilian Ilse, Jakub Tomczak, and Max Welling. Attention-based deep multiple in stance learning. In Jennifer Dy and Andreas Krause, editors, Proceedings of the 35th In ternational Conference on Machine Learning, volume 80 of Proceedings of Machine Learn ing Research, pp. 2127-2136. PMLR, 10-15 Jul. 2018.
He et al. 2015. Kaiming He, Xiangyu Zhang, Shaoqing Ren, and Jian Sun. Deep residual learning for image recognition. CoRR, abs/1512.03385, 2015.
Wang et al. 2021. Bo Wang, Aziz Mezlini, Feyyaz Demir, Marc Fiume, Zhuowen Tu, Michael Brudno, Ben jamin Haibe-Kains, and Anna Goldenberg. SNFtool: Similarity Network Fusion, 2021. R package version 2.3.1.
Ferwerda et al. 2017. Jeremy Ferwerda, Jens Hainmueller, and Chad J. Hazlett. Kernel-based regularized least squares in R (KRLS) and Stata (krls). Journal of Statistical Software, 79(3):1-26, 2017.
Kuijer, 2022. Marieke Lydia Kuijjer. lionessR: Modeling networks for individual samples using LI ONESS, 2022. R package version 1.0.
Karatzoglou et al. 2004. Alexandros Karatzoglou, Alex Smola, Kurt Hornik, and Achim Zeileis. kernlab-an s4 package for kernel methods in r. Journal of statistical software, 11(9):1-20, 2004.
Ritchie et al. 2015. Matthew E Ritchie, Belinda Phipson, DI Wu, Yifang Hu, Charity W Law, Wei Shi, and Gordon K Smyth. limma powers differential expression analyses for ma-sequencing and microarray studies. Nucleic acids research, 43(7):e47-e47, 2015.
Ben Guebila et al. 2022. Marouen Ben Guebila, Tian Wang, Camila M Lopes-Ramos, Viola Fanfani, Deborah Weighill, Rebekka Burkholz, Daniel Schlauch, Joseph N Paulson, Michael AI tenbuchinger, Abhijeet Sonawane, et al. The network zoo: a multilingual package for the inference and analysis of biological networks. bioRxiv, 2022.
Sergushichev 2016. Alexey Sergushichev. An algorithm for fast preranked gene set enrichment analysis us ing cumulative statistic calculation. bioRxiv, 2016.
Liberzon et al. 2015. Arthur Liberzon, Chet Birger, Helga Thor valdsd'ottir, Mahmoud Ghandi, Jill P. Mesirov, and Pablo Tamayo. The molecular signatures database hallmark gene set collec tion. Cell Systems, 1(6):417-425, December 2015.
Blum et al. 2008. Craig Blum, Amanda Graham, Matt Youse fzadeh, Jessica Shrout, Katie Benjamin, Murli Krishna, Raza Hoda, Rana Hoda, David J Cole, Elizabeth Garrett-Mayer, et al. The expression ratio of map7/b2m is prog nostic for survival in patients with stage ii colon cancer. International journal of oncol ogy, 33(3):579-584, 2008.
Hengel et al. 2018. Holger Hengel, Reinhard Keimer, Werner Deigendesch, Angelika RieB, Hiyam Mar zouqa, Jimmy Zaidan, Peter Bauer, and Ludger Schöls. Gpt2 mutations cause devel opmental encephalopathy with microcephaly and features of complicated hereditary spas tic paraplegia. Clinical Genetics, 94(3-4):356-361, 2018.
Chitale et al. 2009. Dhananjay Chitale, Yixuan Gong, Barry S Taylor, Stephen Broderick, Cameron Bren nan, Romel Somwar, Benjamin Golas, Lu Wang, Noriko Motoi, Janos Szoke, et al. An integrated genomic analysis of lung cancer reveals loss of dusp4 in egfr-mutant tumors. Oncogene, 28(31):2773-2783, 2009.
Chandran et al. 2007. Uma R Chandran, Changqing Ma, Rajiv Dhir, Michelle Bisceglia, Maureen Lyons Weiler, Wenjing Liang, George Michalopou los, Michael Becich, and Federico A Monzon. Gene expression profiles of prostate cancer re veal involvement of multiple molecular path ways in the metastatic process. BMC cancer, 7(1):1-21, 2007.
Shedden et al. 2008. Kerby Shedden, Jeremy M G Taylor, Steve A Enkemann, Ming S Tsao, Timothy J Yeat man, William L Gerald, Steve Eschrich, Igor Jurisica, Seshan E Venkatraman, Matthew Meyerson, et al. Gene expression-based sur vival prediction in lung adenocarcinoma: a multi-site, blinded validation study: Direc tor's challenge consortium for the molecular classification of lung adenocarcinoma. Nature medicine, 14(8):822, 2008.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1. A computer-implemented method of characterising a disease in a patient, the method comprising:
Obtaining, for each of a plurality of individuals comprising the patient, biological data comprising values for a plurality of biological factors;
Generating, for each of the plurality of individuals, one or more individual networks, each individual network comprising a plurality of nodes and edges between pairs of the nodes, wherein each node is indicative of a biological factor in the biological data for an individual, and each edge is indicative of a relationship between a pair of biological factors corresponding to the nodes that the edge connects for the respective individual;
Determining the value of one or more similarity metrics between one or more individual networks generated for the patient and one or more individual networks generated for other individuals in the plurality of individuals; and
Predicting a diagnosis or prognosis for the patient using a machine learning model configured to predict a diagnosis or prognosis of the disease in the patient, wherein the machine learning model has been trained to take as input the values of one or more similarity metrics between individual networks and produces as output a diagnosis or prognosis.

2. The method of claim 1, wherein determining the value of one or more similarity metrics comprises determining the value of one or more similarity matrices each comprising the values of a similarity metric between individual networks of pairs of the plurality of individuals.

3. The method of any preceding claim, wherein the one or more similarity metrics comprise, for each of a plurality of pairs of individual networks, a similarity between edges in the individual networks, a similarity between edges in the individual networks and a similarity between nodes in the individual network, or a similarity combining a similarity between edges in the individual networks and a similarity between nodes in the individual network.

4. The method of any preceding claim, wherein each node in an individual network has a value that is the value of a biological factor in the biological data for the respective individual.

5. The method of any preceding claim, wherein each edge in an individual network has a value that is the product of the values of the nodes that it connects for the respective individual, or the difference between the edge value for a network obtained using the plurality of individuals without or without the respective individual, optionally wherein each edge in an individual network has a value e^{x}ᵢⱼ=N*(e^{α}ᵢⱼ−e^{α-x}ᵢⱼ)+e^{α-x}ᵢⱼ, where e^{α}ᵢⱼ is the weight of an edge between nodes i and j in a network modeled on all N individual of the plurality of individuals and e^{α-x}ᵢⱼ is the weight of that edge in a network modeled on all samples except the respective individual x.

6. The method of any preceding claim, wherein the one or more similarity metrics comprise, for each of a plurality of pairs of individual networks:
a similarity between nodes in the individual networks obtained as a Spearman correlation coefficient, an affinity matrix, or a Gaussian kernel using a distance metric between vectors corresponding to the nodes in the respective individuals, or
a similarity combining a similarity between edges in the individual networks and a similarity between nodes in the individual network obtained as a Spearman correlation coefficient, an affinity matrix, or a Gaussian kernel using a distance metric between vectors corresponding to the nodes in the respective individuals.

7. The method of any preceding claim, wherein the one or more similarity metrics comprise, for each of a plurality of pairs of individual networks, a similarity between nodes in the individual networks obtained as a Spearman correlation coefficient, or a similarity combining a similarity between edges in the individual networks and a similarity between nodes in the individual network obtained as a Spearman correlation coefficient.

8. The method of any preceding claim, wherein the one or more similarity metrics comprise, for each of a plurality of pairs of individual networks:
a similarity between edges in the individual networks obtained as an Euclidian distance, Jaccard distance, edge difference distance, DeltaCon distance, spectral distances, graphlet-based measures, Hamming distance, Shortest path kernel, k-step random walk kernel, graph diffusion distance and Portrait Divergence, or
a similarity combining a similarity between nodes in the individual networks and a similarity between edges in the individual network obtained as an Euclidian distance, Jaccard distance, edge difference distance, DeltaCon distance, spectral distances, graphlet-based measures, Hamming distance, Shortest path kernel, k-step random walk kernel, graph diffusion distance and Portrait Divergence.

9. The method of any preceding claim, wherein the one or more similarity metrics comprise, for each of a plurality of pairs of individual networks, a similarity between edges in the individual networks obtained as an edge difference distance, or a similarity combining a similarity between nodes in the individual networks and a similarity between edges in the individual network obtained as an edge difference distance, optionally wherein an edge difference distance is obtained as the Frobenius norm of the difference between a pair of matrices comprising the values of the edges in the respective individual networks for which a similarity is obtained.

10. The method of any preceding claim, further comprising generating a report of the diagnosis or prognosis of the disease in the patient.

11. The method of any preceding claim, further comprising generating the machine learning model configured to predict a diagnosis or prognosis of the disease in patients.

12. The method of any preceding claim, wherein the biological data for each of a plurality of individuals comprises values for a plurality of biological factors comprising a plurality of sets of factors obtained using respective data modalities, wherein the biological data comprises biological data obtained using a plurality of data modalities.

13. The method of claim 12, wherein the biological data for each of the plurality of individuals comprises values for a plurality of biological factors derived from at least one of transcriptomics, proteomics, metabolomics, microbiome, clinical, medical imaging, demographic or histopathology data, optionally wherein the biological data for each of the plurality of individuals comprises values for a plurality of biological factors derived from transcriptomic or proteomic data and values for a plurality of biological factors obtained from histopathology data.

14. The method of any preceding claim, wherein the obtaining for each of the plurality of individuals, one or more individual networks, comprises obtaining for each of the plurality of individuals at least one individual network using values for a plurality of biological factors that comprise biological factors obtained using at least two different data modalities; and/or
wherein the one or more similarity metrics between one or more individual networks comprise one or more similarity metrics derived from individual networks that are obtained from data comprising values of biological factors obtained using at least two different data modalities.

15. The method of any preceding claim, wherein the obtaining for each of the plurality of individuals, one or more individual networks, comprises obtaining for each of the plurality of individuals, a plurality of individual networks, each individual network being obtained using values for a respective plurality of biological factors, optionally wherein each individual network is obtained using values for a respective plurality of biological factors obtained using the same data modality, and the plurality of individual networks comprise individual networks obtained using at least two different data modalities.

16. The method of any preceding claim, wherein the one or more similarity metrics between one or more individual networks comprise a first set of one or more similarity metrics derived from individual networks that are obtained from data comprising values of biological factors obtained using a first set of data modalities, and a second set of one or more similarity metrics derived from individual networks that are obtained from data comprising values of biological factors obtained using a second set of data modalities, wherein the first set is different from the second set.

17. The method of any preceding claim, wherein the one or more similarity metrics between one or more individual networks comprise one or more similarity metrics obtained by combining, for a pair of individuals, a plurality of similarity metrics each derived from a pair of individual networks for the respective individuals obtained from data comprising values of biological factors obtained using a different set of one or more data modalities.

18. The method of any preceding claim, wherein the machine learning model comprises a plurality of machine learning models, each machine learning model configured to predict a diagnosis or prognosis of the disease in the patient, wherein each machine learning model has been trained to take as input the values of a respective subset of the one or more similarity metrics between individual networks and produce as output a diagnosis or prognosis, wherein the respective subsets of similarity metrics are derived from individual networks that are generated from values of biological factors obtained using respective data modalities, and wherein providing a diagnosis or prognosis for the patient comprises combining the outputs of the plurality of machine learning models.

19. The method of any preceding claim, wherein the machine learning model comprises a classification or a regression model, and/or wherein the machine learning model comprises a support vector machine model.

20. The method of any preceding claim, wherein providing a diagnosis or prognosis for the patient comprises combining predicting a disease subtype or severity, and/or wherein the disease is cancer.

21. The method of any preceding claim, wherein providing a diagnosis or prognosis for the patient comprises:
predicting a Gleason score for a patient diagnosed as having prostate cancer,
classifying a patient diagnosed as having brain cancer between a first class corresponding to brain lower grade glioma (Igg) and a second class corresponding to gliobastoma multiforme (gbm), or
classifying a patient diagnosed as having lung cancer between a first class corresponding to lung adenocarcinoma (luad) and a second class corresponding to lung squamous call carcinoma (lusc).

22. The method of any preceding claim, wherein the biological factors comprise gene or protein expression levels and optionally histopathology data, and wherein:
the disease is prostate cancer and the biological factors comprises an expression level for MAP7;
the disease is brain cancer and the biological factors comprises an expression level for GTP2 and/or HIPK2; or
the disease is lung cancer and the biological factors comprises an expression level for TGM2 and/or DUSP4.

23. The method of any preceding claim, wherein the biological factors comprise latent variables of a trained machine learning model applied to image data, optionally wherein the image data is histopathology data and/or wherein the trained machine learning model is a machine learning model, optionally a neural network, that has been trained in a supervised manner to take as input histopathology data and provide as output a disease type label.

24. The method of any preceding claim, wherein at least one of the one or more individual networks, optionally all of the one or more individual networks, comprises nodes that have been selected using a feature selection process and/or edges that have been selected using a feature selection process, and/or wherein generating, for each of the plurality of individuals, one or more individual networks comprises applying a feature selection process to a plurality of nodes each indicative of a biological factor in the biological data for an individual, and/or applying a feature selection process to a plurality of edges is indicative of a relationship between a pair of biological factors corresponding to the nodes that the edge connects for the respective individual.

25. The method of any preceding claim, wherein generating, for each of the plurality of individuals, one or more individual networks comprises selecting a plurality of nodes each indicative of a biological factor in the biological data for an individual for inclusion in each respective individual network, wherein the selection is performed separately for each individual or collectively for the plurality of individuals, optionally wherein selecting a plurality of nodes comprises selecting a plurality of biological factors that are different between an individual and a reference set of individuals or selecting a plurality of nodes that have a variability across the plurality of individuals that satisfies one or more predetermined criteria.

26. The method of any preceding claim, wherein generating, for each of the plurality of individuals, one or more individual networks comprises selecting a plurality of edges indicative of a relationship between a pair of biological factors corresponding to the nodes that the edge connects for the respective individual, wherein the selection is performed separately for each individual or collectively for the plurality of individuals, optionally wherein selecting a plurality of edges comprises selecting a plurality of edges that are different between an individual and a reference set of individuals or selecting a plurality of edges that are different between a plurality of subsets of the plurality of individuals.

27. The method of any preceding claim, wherein generating, for each of the plurality of individuals, one or more individual networks comprises selecting a plurality of nodes each indicative of a biological factor in the biological data for an individual for inclusion in each respective individual network, wherein selecting a plurality of nodes comprises selecting a plurality of nodes that have a variability across the plurality of individuals that satisfies one or more predetermined criteria, and/or
wherein generating, for each of the plurality of individuals, one or more individual networks comprises selecting a plurality of edges indicative of a relationship between a pair of biological factors corresponding to the nodes that the edge connects for the respective individual, wherein selecting a plurality of edges comprises selecting a plurality of edges that are associated with a difference between (a) a first edge value obtained for a pair of nodes for a first subset of the plurality of individuals, and (b) a second edge value obtained for the same pair of nodes for a second subset of the plurality of individuals, the difference satisfying a predetermined criterion, optionally wherein the first edge value is the correlation between the pair of nodes across the first subset of the plurality of individuals and the second edge value is the correlation between the pair of nodes across the second subset of the plurality of individuals and/or wherein the predetermined criterion is the difference being amongst a predetermined threshold or amongst the top x differences amongst all possible edges between nodes in the individual networks, optionally after node selection.

28. A computer-implemented method for obtaining a tool for characterising a disease in a patient, the method comprising:
Obtaining, for each of a plurality of training individuals biological data comprising values for a plurality of biological factors for the individual, and a diagnosis or prognosis label associated with the individual;
Generating, for each of the plurality of individuals, one or more individual networks, each individual network comprising a plurality of nodes and edges between pairs of the nodes, wherein each node is indicative of a biological factor in the biological data for an individual, and each edge is indicative of a relationship between a pair of biological factors corresponding to the nodes that the edge connects for the respective individual;
Determining the value of one or more similarity metrics between one or more individual networks generated for the patient and one or more individual networks generated for other individuals in the plurality of individuals; and
Generating a machine learning model configured to predict a diagnosis or prognosis of the disease in a patient, wherein the machine learning model takes as input the values of the one or more similarity metrics between individual networks and produces as output a diagnosis or prognosis.

29. A computer-implemented method for providing a treatment recommendation for a patient with a disease, the method comprising:
Characterising the disease in the patient using the method of any of claims 1 to 27, and
Selecting the patient for treatment with a treatment associated with the predicted diagnosis or prognosis.

30. A computer-implemented method of performing quality control for biological data about a patient with a disease, the method comprising:
Characterising the disease in the patient using the method of any of claims 1 to 27 using biological data about the patient comprising values for a plurality of subsets of biological factors obtained using respective different data modalities;
Characterising the disease in the patient using the method of any of claims 1 to 27 using biological data about the patient comprising only values for a first subset of biological factors; and
Comparing the predicted diagnosis or prognosis obtained using the plurality of subsets of biological factors and the first subset of biological factors, wherein a predicted diagnosis or prognosis being different for the first subset of biological factors compared to the plurality of subsets of biological factors is indicative of poor quality of the biological data comprising the first subset of biological factors.

31. A system comprising:
at least one processor; and
at least one non-transitory computer readable medium containing instructions that, when executed by the at least one processor, cause the at least one processor to perform the method of any of claims 1 to 30.

32. A non-transitory computer readable medium containing instructions that, when executed by at least one processor, cause the at least one processor to perform the method of any of claims 1 to 30.
